(19) United States
(12) Patent Application Publication
(10) Pub. No.: US 2023/0310319 A1
(43) Pub. Date: Oct. 5, 2023

(54) CULTIVATION OF PLACENTA TO ISOLATE EXOSOMES

(71) Applicant: Celularity Inc., Florham Park, NJ (US)

(72) Inventor: Qian YE, Martinsville, NJ (US)

(73) Assignee: Celularity Inc., Florham Park, NJ (US)

(21) Appl. No.: 18/205,303

(22) Filed: Jun. 2, 2023

Related U.S. Application Data
(63) Continuation of application No. 16/194,278, filed on Nov. 16, 2018, now abandoned.

(51) Int. Cl. A61K 9/127; A61P 35/00; A61K 9/00; A61K 31/7105; A61K 35/17; A61K 35/22; A61K 35/50; A61K 38/17; C12N 5/073; C12N 5/0781; C12N 5/0783; C12N 5/0789; C12N 5/071; C12N 5/077; C12N 5/09

(52) U.S. Cl. CPC A61K 9/127; A61P 35/00; A61K 9/0014; A61K 9/0019; A61K 31/7105; A61K 35/17; A61K 35/22; A61K 35/50; A61K 38/1793; C12N 5/0605; C12N 5/0635; C12N 5/0636; C12N 5/0647; C12N 5/069; C12N 5/0656; C12N 5/0693; A61K 45/06

(57) ABSTRACT

Several approaches to produce, isolate, and characterize exosomes recovered from a cultivated placenta or a portion thereof are provided. The alternatives described herein facilitate the production, isolation, and characterization of exosomes, which can be used as biotechnological tools and therapeutics. Also provided herein are populations of exosomes derived from placenta organ culture or culture of portions of the placenta. Also provided are compositions comprising the populatons of exosomes and methods of their use for the treatment of subjects.

This application claims benefit of U.S. Provisional Patent Application No. 62/587,335, filed Nov. 16, 2018, the disclosure of which is incorporated by reference herein in its entirety.

### 1. FIELD OF THE INVENTION

Methods to produce, isolate, and characterize exosomes from a cultivated placenta or a portion thereof are provided. The alternatives described herein facilitate the production, isolation, and characterization of exosomes, which can be used as biotechnological tools and therapeutics.

### 2. BACKGROUND OF THE INVENTION

Exosomes are nano-sized bi-lipid membrane vesicles secreted from living cells, which play important functions in cell-cell communications. During human pregnancy, the placenta plays a central role in regulating physiological homeostasis and supporting fetal development. It is known that extracellular vesicles and exosomes secreted by placenta contribute to the communication between placenta and maternal tissues to maintain maternal-fetal tolerance. Exosomes contain active biologics including lipids, cytokines, microRNA, mRNA and DNA, as well as, proteins, which can be presented on the surface of the exosomes. Exosomes are thought to be useful for many therapeutic approaches including immune modulation, the promotion of angiogenesis, and for the delivery of medicaments. The need for more approaches that allow for the isolation of large quantities of exosomes is manifest.

### 3. SUMMARY

Aspects of the present invention concern methods to produce, isolate, and characterize exosomes from a cultivated placenta or a portion thereof. The approaches described herein facilitate the production, isolation, and characterization of exosomes, which can be used as biotechnological tools and therapeutics. Preferred alternatives include:
1. A method of exosome isolation from a placenta or a portion thereof, the method comprising:
a) contacting a placenta or a portion thereof, preferably cultured placenta or a portion thereof, with a first medium; and
b) obtaining a first fraction comprising a population of exosomes from said placenta or portion thereof;
c) optionally, contacting said placenta or portion thereof with a second medium and obtaining a second fraction comprising a population of exosomes from said placenta or portion thereof;
d) optionally, contacting said placenta or portion thereof with a third medium and obtaining a third fraction comprising a population of exosomes from said placenta or portion thereof; and
e) optionally, isolating the population of exosomes from said first, second, and/or third fractions, preferably by sequential centrifugation and/or affinity chromatography using antibodies or a binding portion thereof specific for a marker or peptide present on a desired population of exosomes, wherein said antibodies or a binding portion thereof are immobilized on a substrate such as a membrane, a resin, a bead, or a vessel.
2. The method of alternative 1, wherein the placenta or portion thereof further comprises amniotic membrane.
3. The method of alternative 2, wherein the placenta or a portion thereof is a human placenta or a portion thereof.
4. The method of any one of the aforementioned alternatives, wherein the first, second, and/or third mediums are in contact with the placenta or portion thereof for at least 45 minutes, such as 45 minutes or 1, 2, 3, 4, 5, 6, 7, 8, 9, 10, 11, 12, 13, 14, 15, 16, 17, 18, 19, 20, 21, 22, 23, or 24 hours or 2, 3, 4, 5, 6, 7, 8, 9, 10, 11, 12, 13, 14, 15, 16, 17, 18, 19, or 20 days any amount of time that is within a range defined by any two of the aforementioned time points.
5. The method of any one of the aforementioned alternatives, wherein the first, second, and/or third mediums are in contact with the placenta or portion thereof for at least 7, 14, 28, 35 or 42 days or any amount of time that is within a range defined by any two of the aforementioned time points.
6. The method of any one of the aforementioned alternatives, wherein the placenta or portion thereof has been minced, ground, or enzymatically treated.
7. The method of any one of alternatives 1-5, wherein said placenta or portion thereof is substantially flat or sheet-like and has been decellularized and substantially dried, and wherein the method further comprises contacting a fluid comprising exogenous cells with the decellularized placenta or portion thereof so as to seed the decellularized placenta or portion thereof with said exogenous cells and, wherein the contacting of the cells with the decellularized placenta or portion thereof has been performed prior to contacting the decellularized placenta or portion thereof with a first medium.
8. The method of alternative 7, wherein said exogenous cells are obtained from a subject different than the donor subject of said placenta or portion thereof
9. The method of alternative 7 or 8 wherein the fluid comprises is ascites fluid, blood or plasma.
10. The method of alternative 7 or 8, wherein the cells are from an organ.
11. The method of alternative 10, wherein the cells are from liver, kidney, lung or pancreas.
12. The method of alternative 7 or 8, wherein the cells are immune cells.
13. The method of alternative 12, wherein the cells are T-cells or B-cells.
14. The method of any one of the aforementioned alternatives, wherein the first medium comprises Phosphate buffered saline (PBS).
15. The method of alternative 9, wherein the first, second, or third fractions comprise exosomes from ascites fluid, blood or plasma.
16. The method of alternative 10, wherein the first, second, or third fractions comprise exosomes from an organ cell.
17. The method of alternative 11, wherein the cell is from liver, kidney, lung or pancreas.
18. The method of any one of the aforementioned alternatives, wherein the second medium comprises growth factors.
19. The method of any one of the aforementioned alternatives, wherein the third medium comprises a chelator.
20. The method of alternative 19, wherein the chelator is a phosphonate, BAPTA tetrasodium salt, BAPTA/AM, Di-Notrophen TM reagent tetrasodium salt, EGTA/AM, pyridoxal isonicotinoyl hydrazine, N,N,N′,N′-tetrakis-(2 Pyridylmethyl)ethylenediamine, 6-Bromo-N-(2-hydroxybenzylidene)-2-methylquinoline-4-carbohydrazide, 1,2-Bis(2-aminophenoxy)ethane-N,N,N,N′-tetraacetic acid tetrakis(acetoxymethyl ester), (Ethyl enedinitrilo)tetraacetic acid, (EDTA), Edathamil, Ethyl enedinitrilotetraacetic acid, Ethylene glycol-bis(2-aminoethylether)-N,N,N′,N′-tetraacetic acid, or Ethylene glycol-bis(β-aminoethyl ether)-N,N,N′,N′-tetraacetic acid tetrasodium salt (EGTA) or any combination thereof.
21. The method of any one of alternatives 19 or 20, wherein the chelator is EDTA or EGTA or a combination thereof.
22. The method of any one of alternatives 19-21, wherein the chelator is provided in the third medium at a concentration of 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM or 100 mM or at a concentration that is within a range defined by any two aforementioned concentrations.
23. The method of any one of alternatives 19-22, wherein the concentration of EDTA in the third medium is provided at a concentration of 1 mM, 2 mM, 3 mM, 4 mM, 5 mM, 6 mM, 7 mM, 8 mM, 9 mM 10 mM, 20 mM, 30 mM, 40 mM, 50 mM, 60 mM, 70 mM, 80 mM, 90 mM or 100 mM or at a concentration that is within a range defined by any two aforementioned concentrations.
24. The method of any one of the aforementioned alternatives, wherein the third medium comprises a protease.
25. The method of alternative 24, wherein the protease is a trypsin, collagenase, chymotrypsin or carboxypeptidase or any combination thereof
26. The method of alternative 25 or 25, wherein the protease is trypsin.
27. The method of alternative 24, wherein the protease is provided in the third medium is provided at a concentration of 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM or 100 mM or at a concentration that is within a range defined by any two of the aforementioned concentrations.
28. The method of any one of the aforementioned alternatives, wherein the method further comprises contacting the placenta or portion thereof with an additional plurality of mediums, wherein the contacting results in obtaining multiple fractions comprising exosomes.
29. The method of alternative 28, wherein the first, second, third or additional mediums comprise glucose.
30. The method of alternative 28 or 29, wherein the first, second, third or additional mediums comprise GM-CSF.
31. The method of any one of alternatives 28-30, wherein the first, second, third or additional mediums comprise serum.
32. The method of any one of alternatives 28-31, wherein the first, second, third or additional mediums comprise DMEM.
33. The method of any one of alternatives 28-32, wherein the first, second, third or additional medium comprises an AHR antagonist.
34. The method of alternative 33, wherein the AHR antagonist is SR1.
35. The method of alternative 34, wherein the SR1 is at a concentration of 1 nM, 10 nM, 100 nM, 200 nM, 300 nM, 400 nM, 500 nM, 600 nM, 700 nM, 800 nM, 900 nM or 1 mM or any other concentration within a range defined by any two aforementioned values.
36. The method of any one of the aforementioned alternatives, wherein the first medium is in contact with the placenta or portion thereof while maintaining a temperature of 0° C., 5° C., 10° C., 15° C., 20° C., 25° C., 30° C., 35° C. or 40° C. or a temperature that is within a range defined by any two of the aforementioned temperatures.
37. The method of any one of the aforementioned alternatives, wherein the second medium is in contact with the placenta or portion thereof while maintaining a temperature of 0° C., 5° C., 10° C., 15° C., 20° C., 25° C., 30° C., 35° C. or 40° C. or a temperature that is within a range defined by any two of the aforementioned temperatures.
38. The method of any one of the aforementioned alternatives, wherein the third medium is in contact with the placenta or portion thereof while maintaining a temperature of 0° C., 5° C., 10° C., 15° C., 20° C., 25° C., 30° C., 35° C. or 40° C. or a temperature that is within a range defined by any two of the aforementioned values.
39. The method of any one of alternatives 28-38, wherein the additional plurality of mediums is in contact with the placenta or portion thereof while maintaining a temperature of 0° C., 5° C., 10° C., 15° C., 20° C., 25° C., 30° C., 35° C. or 40° C. or a temperature that is within a range defined by any two of the aforementioned values.
40. The method of any one of the aforementioned alternatives, wherein the first, second or third perfusion or additional plurality of mediums comprise antibiotics.
41. The method of any one of the aforementioned alternatives, wherein the exosomes are isolated from said first, second, and/or third fractions or multiple fractions by a method comprising:
(a) passing the first, second and/or third fractions or multiple fractions through a tissue filter;
(b) performing a first centrifugation of the filtrate collected in (a) to generate a cell pellet and a first supernatant;
(c) performing a second centrifugation on the first supernatant to generate a second supernatant; and
(d) performing a third centrifugation on the second supernatant to generate an exosome pellet; and, optionally,
(e) resuspending the exosomes in a solution.
42. The method of any one of the aforementioned alternatives, wherein the exosomes comprise CD63, CD63-A, perforin, Fas, TRAIL or granzyme B or any combination thereof.
43. The method of alternative 42, wherein the exosomes comprise CD63A.
44. The method of any one of the aforementioned alternatives, wherein the exosomes comprise a signaling molecule.
45. The method of any one of the aforementioned alternatives, wherein the exosomes comprise cytokines, mRNA or miRNA.
46. The method of any one of the aforementioned alternatives, further comprising isolating exosomes by affinity chromatography, wherein affinity chromatography is selective for the removal of exosomes comprising viral antigens, viral proteins, bacterial antigens, bacterial proteins, fungal antigens or fungal proteins.
47. The method of any one of the aforementioned alternatives, further comprising isolating exosomes by one or more additional affinity chromatography steps, wherein the one or more additional chromatography step is selective for the removal of exosomes comprising an inflammatory marker and/or a tumor marker.

Also provided is a composition comprising exosomes derived from human placenta, wherein said exosomes are positive for CD1c, CD20, CD24, CD25, CD29, CD2, CD3, CD8, CD9, CD11c, CD14, CD19, CD31, CD40, CD41b, CD42a, CD44, CD45, CD49e, CD4, CD56, CD62P, CD63, CD69, CD81, CD86, CD105, CD133-1, CD142, CD146, CD209, CD326, HLA-ABC, HLA-DRDPDQ MCSP, ROR1, SSEA-4, or combinations thereof.

The exosomes described herein comprise particular markers. Such markers can, for example, be useful in the identification of the exosomes and for distinguishing them from other exosomes, e.g., exosomes not derived from placenta. In certain embodiments, such exosomes are positive for one or more markers, e.g., as determinable by flow cytometry, for example, by fluorescence-activated cell sorting (FACS). In addition, the exosomes provided herein can be identified based on the absence of certain markers. Determination of the presence or absence of such markers can be accomplished using methods known in the art, e.g., fluorescence-activated cell sorting (FACS).

In some embodiments, the exosomes are positive for CD1c, CD20, CD24, CD25, CD29, CD2, CD3, CD8, CD9, CD11c, CD14, CD19, CD31, CD40, CD41b, CD42a, CD44, CD45, CD49e, CD4, CD56, CD62P, CD63, CD69, CD81, CD86, CD105, CD133-1, CD142, CD146, CD209, CD326, HLA-ABC, HLA-DRDPDQ, MCSP, ROR1, and SSEA-4. In some embodiments, the exosomes are positive for 2, 3, 4, 5, 6, 7, 8, 9, 10, or more markers selected from the group consisting of CD1c, CD20, CD24, CD25, CD29, CD2, CD3, CD8, CD9, CD11c, CD14, CD19, CD31, CD40, CD41b, CD42a, CD44, CD45, CD49e, CD4, CD56, CD62P, CD63, CD69, CD81, CD86, CD105, CD133-1, CD142, CD146, CD209, CD326, HLA-ABC, HLA-DRDPDQ, MCSP, ROR1, and SSEA-4.

In some embodiments, the exosomes are CD3-, CD11b-, CD14-, CD19-, CD33-, CD192-, HLA-A-, HLA-B-, HLA-C-, HLA-DR-, CD11c- or CD34-. In some embodiments, the exosomes are CD3-, CD11b-, CD14-, CD19-, CD33-, CD192-, HLA-A-, HLA-B-, HLA-C-, HLA-DR-, CD11c- and CD34-.

In some embodiments, the exosomes comprise non-coding RNA molecules. In some embodiments, the RNA molecules are microRNAs. In some embodiments, the microRNAs are selected from the group consisting of the microRNAs in Table 7, and combinations thereof. In some embodiments, the microRNAs are selected from the group consisting of hsa-mir-26b, hsa-miR-26b-5p, hsa-mir-26a-2, hsa-mir-26a-1, hsa-miR-26a-5p, hsa-mir-30d, hsa-miR-30d-5p, hsa-mir-100, hsa-miR-100-5p, hsa-mir-21, hsa-miR-21-5p, hsa-mir-22, hsa-miR-22-3p, hsa-mir-99b, hsa-miR-99b-5p, hsa-mir-181a-2, hsa-mir-181a-1, hsa-miR-181a-5p, and combinations thereof.

In some embodiments, the exosomes comprise a cytokine selected from the group consisting of the cytokines in Table 3, and combinations thereof.

In some embodiments, the exosomes comprise a cytokine receptor selected from the group consisting of the cytokine receptors in Table 4, and combinations thereof.

In some embodiments, the exosomes comprise a protein selected from the group consisting of the proteins in Table 6, and combinations thereof. In some embodiments, the exosomes comprise a protein selected from the group consisting of Cytoplasmic aconitate hydratase, Cell surface glycoprotein MUC18, Protein arginine N-methyltransferase 1, Guanine nucleotide-binding protein G(s) subunit alpha, Cullin-5, Calcium-binding protein 39, Glucosidase 2 subunit beta, Chloride intracellular channel protein 5, Semaphorin-3B, 60S ribosomal protein L22, Spliceosome RNA helicase DDX39B, Transcriptional activator protein Pur-alpha, Programmed cell death protein 10, BRO1 domain-containing protein BROX, Kynurenine-oxoglutarate transaminase 3, Laminin subunit alpha-5, ATP-binding cassette sub-family E member 1, Syntaxin-binding protein 3, Proteasome subunit beta type-7, and combinations thereof.

In some embodiments, the exosomes comprise at least one marker molecule at a level at least two-fold higher than exosomes derived from mesenchymal stem cells, cord blood, or placental perfusate. In some embodiments, the exosomes comprise at least one marker molecule at a level at least two-fold higher than exosomes derived from mesenchymal stem cells, cord blood, and placental perfusate.

In some embodiments, the exosomes are isolated from media of a whole placenta culture. In some embodiments, the exosomes are isolated from media of a whole culture comprising placental lobes or portions of a placenta.

In some embodiments, the exosomes are produced by the methods of the invention. In some embodiments, the composition is in a form suitable for intravenous administration. In some embodiments, the composition is in a form suitable for local injection. In some embodiments, the composition is in a form suitable for topical administration. In some embodiments, the composition is in a form suitable for ultrasonic delivery.

Also provided are methods of increasing the proliferation of an immune cell comprising contacting the cell with a composition of any one of claims 48-65.

In some embodiments the immune cell is a T cell.
In some embodiments the immune cell is an NK cell.
In some embodiments the immune cell is a CD34+ cell.

Also provided are methods of inhibiting the proliferation of a cancer cell comprising contacting the cell with a composition of the invention.

Also provided are methods of angiogenesis or vascularization in said subject comprising administering the composition of the invention to the subject.

Also provided are methods of modulating the immune system of a said subject comprising administering the composition of the invention to the subject.

Also provided are methods of repairing diseased or damages tissue in a subject comprising administering the composition of the invention to the subject.

Also provided are methods of treating a cancer in a subject comprising administering the composition of the invention to the subject.

In some embodiments of the above methods, the subject is human.

Also provided herein are compositions comprising exosomes. Such compositions generally do not comprise placental cells from which the exosomes have been derived. Moreover, such compositions generally do not comprise cell culture supernatant from the cell culture from which the exosomes have been derived.

In certain embodiments, purified exosomes are formulated into pharmaceutical compositions suitable for administration to a subject in need thereof. In certain embodiments, said subject is a human. The placenta-derived exosome-containing pharmaceutical compositions provided herein can be formulated to be administered locally, systemically subcutaneously, parenterally, intravenously, intramuscularly, topically, orally, intradermally, transdermally, or intranasally to a subject in need thereof. In a certain embodiment, the placenta-derived exosome-containing pharmaceutical compositions provided herein are formulated for local administration. In a certain embodiment, the placenta-derived exosome-containing pharmaceutical compositions provided herein are formulated for systemic subcutaneous administration. In a certain embodiment, the placenta-derived exosome-containing pharmaceutical compositions provided herein are formulated for parenteral administration. In a certain embodiment, the placenta-derived exosome-containing pharmaceutical compositions provided herein are formulated for intramuscular administration. In a certain embodiment, the placenta-derived exosome-containing pharmaceutical compositions provided herein are formulated for topical administration. In a certain embodiment, the placenta-derived exosome-containing pharmaceutical compositions provided herein are formulated for oral administration. In a certain embodiment, the placenta-derived exosome-containing pharmaceutical compositions provided herein are formulated for intradermal administration. In a certain embodiment, the placenta-derived exosome-containing pharmaceutical compositions provided herein are formulated for transdermal administration. In a certain embodiment, the placenta-derived exosome-containing pharmaceutical compositions provided herein are formulated for intranasal administration. In a specific embodiment, the placenta-derived exosome-containing pharmaceutical compositions provided herein are formulated for intravenous administration.

In another aspect, provided herein are uses of the exosomes and/or pharmaceutical compositions comprising exosomes described herein.

In a specific embodiment, the exosomes and/or pharmaceutical compositions comprising exosomes described herein are used to treat and/or prevent diseases and/or conditions in a subject in need thereof. In a specific embodiment, the exosomes and/or pharmaceutical compositions comprising exosomes described herein are used to promote angiogenesis and/or vascularization in a subject in need thereof. In another specific embodiment, the exosomes and/or pharmaceutical compositions comprising exosomes described herein are used to modulate immune activity (e.g., increase an immune response or decrease an immune response) in a subject in need thereof. In another specific embodiment, the exosomes and/or pharmaceutical compositions comprising exosomes described herein are used to repair tissue damage, e.g., tissue damage caused by an acute or chronic injury, in a subject in need thereof.

In another specific embodiment, the derived exosomes and/or pharmaceutical compositions comprising exosomes described herein are for use in a method for treating and/or preventing diseases and/or conditions in a subject in need thereof. In another embodiment, the pharmaceutical compositions comprising exosomes described herein are for use in a method for treating diseases and/or conditions in a subject in need thereof. In another embodiment, the pharmaceutical compositions comprising exosomes described herein are for use in a method for preventing diseases and/or conditions in a subject in need thereof. In a specific embodiment, the pharmaceutical compositions comprising exosomes described herein are for use in a method for promoting angiogenesis and/or vascularization in a subject in need thereof. In another specific embodiment, the pharmaceutical compositions comprising exosomes described herein are for use in a method for modulating immune activity (e.g., increase an immune response or decrease an immune response) in a subject in need thereof. In another specific embodiment, the pharmaceutical compositions comprising exosomes described herein are for use in a method for repairing tissue damage, e.g., tissue damage caused by an acute or chronic injury, in a subject in need thereof.

In another specific embodiment, the exosomes and/or pharmaceutical compositions comprising exosomes described herein are used as cytoprotective agents. In another aspect, the exosomes and/or pharmaceutical compositions comprising exosomes described herein are provided in the form of a kit suitable for pharmaceutical use.

### 4. BRIEF DESCRIPTION OF THE DRAWINGS

FIG. 1 shows a schematic for cultivating cells for exosome isolation.

FIG. 2A-FIG. 2C show three pExo isolates that were analyzed for their size distribution by NanoSight. This work was performed and reported by SBI Inc. (System Bioscience Inc.) using a contract service (www.systembio.com/services/exosome-services/).

FIG. 3A-FIG. 3C show protein markers present on pExo (N=12) (FIG. 3A) compared with placenta perfusate exosomes (FIG. 3B) and cord blood serum derived exosomes (FIG. 3C) using the MACSPlex Kit.

FIG. 4 shows functional pathways of proteins identified in placental exosome populations.

FIG. 5 shows common and unique protein identified in three placenta exosome samples.

FIG. 6 shows that pExo promote migration of human dermal fibroblast cells in a transwell system.

FIG. 7 shows that pExo promote migration of human umbicical cord vessel endothelial cells.

FIG. 8 shows that pExo stimulate the proliferation of HUVEC.

FIG. 9 shows that pExo stimulate the proliferation of human CD34+ cells.

FIG. 10 shows that pExo stimulate the colony formation of human CD34+ cells.

FIG. 11 shows that pExo inhibit the proliferation of SKOV3 cancer cells.

FIG. 12 shows that pExo inhibit the proliferation of A549 cancer cells.

FIG. 13 shows that pExo inhibit the proliferation of MDA321 cancer cells.

FIG. 14 shows that pExo does not affect the proliferation of CD3+ T cells in culture.

FIG. 15 shows that pExo increases expression of activation marker CD69 in UBC T CD3+ cells.

FIG. 16 shows that pExo increases expression of activation marker CD69 in adult PBMC T CD3+ cells.

FIG. 17 shows that pExo increases CD56+ NK cells in PBMC.

### 5. DETAILED DESCRIPTION

### 5.1. Placenta-Derived Exosomes

The placenta-derived exosomes described herein can be selected and identified by their morphology and/or molecular markers, as described below. The placenta-derived exosomes described herein are distinct from exosomes known in the art e.g., chorionic villi mesenchymal stem cell-derived exosomes, e.g., those described in Salomon et al., 2013, PLOS ONE, 8:7, e68451. Accordingly, the term “placenta-derived exosome,” as used herein, is not meant to include exosomes obtained or derived from chorionic villi mesenchymal stem cells.

In certain embodiments, populations of placenta-derived exosomes described herein do not comprise cells, e.g., nucleated cells, for example placental cells.

5.1.1. Placenta-Derived Exosome Markers

The placenta-derived exosomes described herein contain markers that can be used to identify and/or isolate said exosomes. These markers may, for example, be proteins, nucleic acids, saccharide molecules, glycosylated proteins, lipid molecules, and may exist in monomeric, oligomeric and/or multimeric form. In certain embodiments, the markers are produced by the cell from which the exosomes are derived. In certain embodiments, the marker is provided by the cell from which the exosomes are derived, but the marker is not expressed at a higher level by said cell. In a specific embodiment, the markers of exosomes described herein are higher in the exosomes as compared to the cell of origin when compared to a control marker molecule. In another specific embodiment, the markers of exosomes described herein are enriched in said exosomes as compared to exosomes obtained from another cell type (e.g., the chorionic villi mesenchymal stem cells described in Salomon et al., 2013, PLOS ONE, 8:7, e68451 and pre-adipocyte mesenchymal stem cells), wherein the exosomes are isolated through identical methods.

The three-dimensional structure of exosomes allows for the retention of markers on the surface of the exosome and/or contained within the exosome. Similarly, marker molecules may exist partially within the exosome, partially on the outer surface of the exosome and/or across the phospholipid bilayer of the exosome. In a specific embodiment, the markers associated with the exosomes described herein are proteins. In certain embodiments, the markers are transmembrane proteins that are anchored within the exosome phospholipid bilayer, or are anchored across the exosome phospholipid bilayer such that portions of the protein molecule are within the exosome while portions of the same molecule are exposed to the outer surface of the exosome. In certain embodiments, the markers are contained entirely within the exosome. In another specific embodiment, the markers associated with the exosomes described herein are nucleic acids. In certain embodiments, said nucleic acids are non-coding RNA molecules, e.g., micro-RNAs (miRNAs).

5.1.1.1. Surface Markers

The exosomes described herein comprise surface markers that allow for their identification and that can be used to isolate/obtain substantially pure populations of cell exosomes free from their cells of origin and other cellular and non-cellular material. Methods of for determining exosome surface marker composition are known in the art. For example, exosomal surface markers can be detected by fluorescence-activated cell sorting (FACS) or Western blotting.

In certain embodiments, the exosomes described herein comprise a surface marker at a greater amount than exosomes known in the art, as determinable by, e.g., FACS.

5.1.1.2. Yeild

The exosomes described herein may be isolated in accordance with the methods described herein and their yields may be quantified. In a specific embodiment, the exosomes described herein are isolated at a concentration of about 0.5-5.0 mg per liter of culture medium (e.g., culture medium with or without serum). In another specific embodiment, the exosomes described herein are isolated at a concentration of about 2-3 mg per liter of culture medium (e.g., culture medium containing serum). In another specific embodiment, the exosomes described herein are isolated at a concentration of about 0.5-1.5 mg per liter of culture medium (e.g., culture medium lacking serum).

5.1.2. Storage and Preservation

The exosomes described herein can be preserved, that is, placed under conditions that allow for long-term storage, or conditions that inhibit degradation of the exosomes.

In certain embodiments, the exosomes described herein can be stored after collection according to a method described above in a composition comprising a buffering agent at an appropriate temperature. In certain embodiments, the exosomes described herein are stored frozen, e.g., at about −20° C. or about −80° C.

In certain embodiments, the exosomes described herein can be cryopreserved, e.g., in small containers, e.g., ampoules (for example, 2 mL vials). In certain embodiments, the exosomes described herein are cryopreserved at a concentration of about 0.1 mg/mL to about 10 mg/mL.

In certain embodiments, the exosomes described herein are cryopreserved at a temperature from about −80° C. to about −180° C. Cryopreserved exosomes can be transferred to liquid nitrogen prior to thawing for use. In some embodiments, for example, once the ampoules have reached about −90° C., they are transferred to a liquid nitrogen storage area. Cryopreservation can also be done using a controlled-rate freezer. Cryopreserved exosomes can be thawed at a temperature of about 25° C. to about 40° C. before use.

In certain embodiments, the exosomes described herein are stored at temperatures of about 4° C. to about 20° C. for short periods of time (e.g., less than two weeks).

### 5.2. Compositions

Further provided herein are compositions, e.g., pharmaceutical compositions, comprising the exosomes provided herein. The compositions described herein are useful in the treatment of certain diseases and disorders in subjects (e.g., human subjects) wherein treatment with exosomes is beneficial.

In certain embodiments, in addition to comprising the exosomes provided herein, the compositions (e.g., pharmaceutical compositions) described herein comprise a pharmaceutically acceptable carrier. As used herein, the term “pharmaceutically acceptable” means approved by a regulatory agency of the Federal or a state government or listed in the U.S. Pharmacopeia or other generally recognized pharmacopeiae for use in animals, and more particularly in humans. The term “carrier,” as used herein in the context of a pharmaceutically acceptable carrier, refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by JP Remington and AR Gennaro, 1990, 18th Edition.

In certain embodiments, the compositions described herein additionally comprise one or more buffers, e.g., saline, phosphate buffered saline (PBS), Dulbecco's PBS (DPBS), and/or sucrose phosphate glutamate buffer. In other embodiments, the compositions described herein do not comprise buffers. In certain embodiments, the compositions described herein additionally comprise plasmalyte.

In certain embodiments, the compositions described herein additionally comprise one or more salts, e.g., sodium chloride, calcium chloride, sodium phosphate, monosodium glutamate, and aluminum salts (e.g., aluminum hydroxide, aluminum phosphate, alum (potassium aluminum sulfate), or a mixture of such aluminum salts). In other embodiments, the compositions described herein do not comprise salts.

The compositions described herein can be included in a container, pack, or dispenser together with instructions for administration.

The compositions described herein can be stored before use, e.g., the compositions can be stored frozen (e.g., at about −20° C. or at about −80° C.); stored in refrigerated conditions (e.g., at about 4° C.); or stored at room temperature.

5.2.1. Formulations and Routes of Administration

The amount of exosomes or a composition described herein which will be effective for a therapeutic use in the treatment and/or prevention of a disease or condition will depend on the nature of the disease, and can be determined by standard clinical techniques. The precise dosage of exosomes, or compositions thereof, to be administered to a subject will also depend on the route of administration and the seriousness of the disease or condition to be treated, and should be decided according to the judgment of the practitioner and each subject's circumstances. For example, effective dosages may vary depending upon means of administration, target site, physiological state of the patient (including age, body weight, and health), whether the patient is human or an animal, other medications administered, and whether treatment is prophylactic or therapeutic. Treatment dosages are optimally titrated to optimize safety and efficacy.

Administration of the exosomes described herein, or compositions thereof can be done via various routes known in the art. In certain embodiments, the exosomes described herein, or compositions thereof are administered by local, systemic, subcutaneous, parenteral, intravenous, intramuscular, topical, oral, intradermal, transdermal, or intranasal, administration. In a specific embodiment, said administration is via intravenous injection. In a specific embodiment, said administration is via subcutaneous injection. In a specific embodiment, said administration is topical. In another specific embodiment, the exosomes, or compositions thereof, are administered in a formulation comprising an extracellular matrix. In another specific embodiment, the exosomes, or compositions thereof, are administered in combination with one or more additional delivery device, e.g., a stent. In another specific embodiment, the exosomes, or compositions thereof, are administered locally, e.g., at or around the site of an area to be treated with said exosomes or compositions, such as hypoxic tissue (e.g., in treatment of ischemic diseases) or draining lymph nodes.

### 5.3. Methods of Use

5.3.1. Treatment of Diseases that Benefit from Angiogenesis

The exosomes described herein, and compositions thereof, promote angiogenesis, and, therefore can be used to treat diseases and disorders that benefit from angiogenesis. Accordingly, provided herein are methods of using the exosomes described herein, or compositions thereof, to promote angiogenesis in a subject in need thereof. As used herein, the term “treat” encompasses the cure of, remediation of, improvement of, lessening of the severity of, or reduction in the time course of, a disease, disorder or condition, or any parameter or symptom thereof in a subject. In a specific embodiment, the subject treated in accordance with the methods provided herein is a mammal, e.g., a human.

In one embodiment, provided herein are methods of inducing vascularization or angiogenesis in a subject, said methods comprising administering to the subject the exosomes provided herein, or a composition thereof. Accordingly, the methods provided herein can be used to treat diseases and disorders in a subject that that benefit from increased angiogenesis/vascularization. Examples of such diseases/conditions that benefit from increased angiogenesis, and therefore can be treated with the exosomes and compositions described herein included, without limitation, myocardial infarction, congestive heart failure, peripheral artery disease, critical limb ischemia, peripheral vascular disease, hypoplastic left heart syndrome, diabetic foot ulcer, venous ulcer, or arterial ulcer.

In one embodiment, provided herein are methods of treating a subject having a disruption of blood flow, e.g., in the peripheral vasculature, said methods comprising administering to the subject the exosomes provided herein, or a composition thereof. In a specific embodiment, the methods provided herein comprise treating a subject having ischemia with the exosomes provided herein, or a composition thereof. In certain embodiments, the ischemia is peripheral arterial disease (PAD), e.g., is critical limb ischemia (CLI). In certain other embodiments, the ischemia is peripheral vascular disease (PVD), peripheral arterial disease, ischemic vascular disease, ischemic heart disease, or ischemic renal disease.

5.3.2. Patient Populations

In certain embodiments, the exosomes described herein are administered to a subject in need of therapy for any of the diseases or conditions described herein. In another embodiment, a composition described herein is administered to a subject in need of therapy for any of the diseases or conditions described herein. In certain embodiments said subject is a human.

In a specific embodiment, the exosomes or compositions described herein are administered to a subject (e.g., a human) in need of a therapy to increase angiogensis and/or vascularization.

### 5.4. Kits

Provided herein is a pharmaceutical pack or kit comprising one or more containers filled with one or more of the ingredients of the pharmaceutical compositions described herein, i.e., compositions comprising the exosomes described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

The kits described herein can be used in the above methods. The compositions described herein can be prepared in a form that is easily administrable to an individual. For example, the composition can be contained within a container that is suitable for medical use. Such a container can be, for example, a sterile plastic bag, flask, jar, or other container from which the compositions can be easily dispensed. For example, the container can be a blood bag or other plastic, medically-acceptable bag suitable for the intravenous administration of a liquid to a recipient.

### Exemplary Placenta Culture

The placenta is a reservoir of cells, including stem cells such as hematopoietic stem cells (HSC) and non-hematopoietic stem cells. Described herein are methods to isolate exosomes from a placenta or portion thereof, which is cultured in a bioreactor. Exosomes are secreted by the cells during the culture and the exosomes are secreted into the media, which facilitates further processing and isolation of the exosomes. Exosomes can be also isolated from the placenta or portion thereof at different stages of culture (e.g., at different time points and different perfusion liquids may be used at each recovery step). Once in the media, the exosomes can be further isolated using e.g., centrifugation, a commercially available exosome isolation kit, lectin affinity, and/or affinity chromatography (e.g., utilizing immobilized binding agents, such as binding agents attached to a substrate, which are specific for a small Rab family GTPase, annexin, flotillin, Alix, Tsg101, ESCRT complex, CD9, CD37, CD53, CD63, CD63A, CD81, CD82), Hsp70, Hsp90, epithelial cell adhesion molecules (EpCam), perforin, TRAIL, granzyme B, Fas, one or more cancer markers such as: Fas ligand, CD24, EpCAM, EDIL3, fibronectin, Survivin, PCA3, TMPRSS2:ERG, Glypican-1, TGF-β1, MAGE 3/6, EGFR, EGFRvIII, CD9, CD147, CA-125, EpCam, and/or CD24, or one or more inflammatory or pathogenic markers such as: a viral, fungal, or a bacterial protein or peptide including but not limited to α-synuclein, HIV or HCV proteins, tau, beta-amyloid, TGF-beta, TNF-alpha, fetuin-A, and/or CD133). The isolated exosomes can be used for therapeutics, diagnostics, and as biotechnological tools.

“Exosomes” as described herein are vesicles that are present in many and perhaps all eukaryotic fluids, including acscites fluid, blood, urine, serum and breast milk. They may also be referred to as extracellular vesicles. Exosomes are bi-lipid membrane vesicles secreted from living cells that play important functions in cell-cell communications. Exosomes are produced by cells, such a stem cells, epithelial cells and a sub-type of exosomes, defined as Matrix-bound nanovesicles (MBVs), was reported to be present in extracellular matrix (ECM) bioscaffolds (non-fluid). The reported diameter of exosomes is between 30 and 100 nm, which is larger than low-density lipoproteins (LDL) but much smaller than, for example, red blood cells. Exosomes can be released from the cell when multivesicular bodies fuse with the plasma membrane or released directly from the plasma membrane.

Exosomes have been shown to have specialized functions and play a key role in processes such as coagulation, intercellular signaling, and waste management. It is known that extracellular vesicles and exosomes secreted by placenta contribute to the communication between placenta and maternal tissues to maintain maternal-fetal tolerance. Exosomes isolated from human placental explants was shown to have immune modulation activities. Stem cell derived exosomes were also shown to reduce neuroinflammation by suppressing the activation of astrocytes and microglia and promote neurogenesis possibly by targeting the neurogenic niche, both which contribute to nervous tissue repair and functional recovery after TBI. (Review Yang et al. 2017, Frontiers in Cellular Neuroscience). Exosomes derived from human embryonic mesenchymal stem cells also promote osteochondral regeneration (Zhang et al. 2016, Osteoarthritis and Cartilage). Exosomes secreted by human placenta that carry functional Fas Ligand and Trail molecules were shown to convey apoptosis in activated immune cells, suggesting exosome-mediated immune privilege of the fetus. (Ann-Christin Stenqvist et al., Journal of Immunology, 2013, 191: doi:10.4049).

Exosomes contain active biologics including lipids, cytokines, microRNA, mRNA and DNA. They may also function as mediators of intercellular communication via genetic material and/or protein transfer. Exosomes may also contain cell-type specific information that may reflect a cell's functional or physiological state. Consequently, there is a growing interest in the development of clinical and biological applications for exosomes.

Accordingly, exosomes isolated from human placenta or a portion thereof using the approaches described herein, optionally including characterization of said exosomes (e.g., by identifying the presence or absence of one or more proteins or markers on the exosomes) can be used to stimulate an immuno-modulation, an anti-fibrotic environment, and/or a pro-regenerative effect. Accordingly, exosomes isolated from human placenta or a portion thereof using the approaches described herein may be selected (e.g., according to markers present or absent on the exosomes), purified, frozen, lyophilized, packaged and/or distributed as a therapeutic product and/or a biotechnological tool.

In some alternatives, it may be beneficial to identify exosomes having tumor markers or peptides, pathogenic markers or peptides, such as viral, fungal, or bacterial markers or peptides, and/or inflammatory markers, such as inflammatory peptides, so that such exosomes can be removed from a population of exosomes (e.g., removal by affinity chromatography with binding molecules such as, antibodies or binding portions thereof, which are specific for such tumor markers or peptides, pathogenic markers or peptides, and/or inflammatory markers or peptides). Accordingly, in some alternatives, for example, a first population of exosomes are isolated from human placenta or a portion thereof by the methods described herein and once the first population of exosomes is isolated this population of exosomes is further processed to remove one or more subpopulations of exosomes using a substrate having an immobilized antibody or binding portion thereof (e.g., a membrane, a resin, a bead, or a vessel having said immobilized antibody or binding portion thereof), wherein the immobilized antibody or binding portion thereof is specific for a marker or peptide present on the subpopulation of exosomes, which are selected for further isolation, such as, one or more tumor markers or peptides, pathogenic markers or peptides, e.g., viral, fungal, or bacterial markers or peptides, and/or inflammatory markers or inflammatory peptides. In some alternatives, a first population of exosomes isolated from human placenta or a portion thereof by the methods described herein are contacted with a substrate having an immobilized antibody or binding portion thereof (e.g., a membrane, a resin, a bead, or a vessel having said immobilized antibody or binding portion thereof), wherein the immobilized antibody or binding portion thereof is specific for one or more cancer markers such as: Fas ligand, CD24, EpCAM, EDIL3, fibronectin, Survivin, PCA3, TMPRSS2:ERG, Glypican-1, TGF-β1, MAGE 3/6, EGFR, EGFRvIII, CD9, CD147, CA-125, EpCam, and/or CD24 so as to isolate a second population of exosomes from the first population of exosomes based on the affinity to the immobilized antibody or binding portion thereof. In some alternatives, a first population of exosomes isolated from human placenta or a portion thereof by the methods described herein are contacted with a substrate having an immobilized antibody or binding portion thereof (e.g., a membrane, a resin, a bead, or a vessel having said immobilized antibody or binding portion thereof), wherein the immobilized antibody or binding portion thereof is specific for one or more inflammatory or pathogenic markers such as: a viral, fungal, or a bacterial protein or peptide including but not limited to α-synuclein, HIV or HCV proteins, tau, beta-amyloid, TGF-beta, TNF-alpha, fetuin-A, and/or CD133 or portions thereof so as to isolate a second population of exosomes from the first population of exosomes based on the affinity to the immobilized antibody or binding portion thereof.

In some alternatives, the population of exosomes isolated and/or selected by the approaches described herein have markers or peptides that are useful for therapeutics such as perforin and/or granzyme B, which has been shown to mediate anti-tumor activity both in vitro and in vivo (J Cancer 2016; 7(9):1081-1087) or Fas, which has been found in exosomes that exert cytotoxic activity against target cancer cells. (Theranostics 2017; 7(10):2732-2745). Accordingly, in some alternatives, a first population of exosomes isolated from human placenta or a portion thereof by the methods described herein are contacted with a substrate having an immobilized antibody or binding portion thereof (e.g., a membrane, a resin, a bead, or a vessel having said immobilized antibody or binding portion thereof), wherein the immobilized antibody or binding portion thereof is specific for perforin, TRAIL and/or granzyme B and/or Fas and a second population of exosomes from the first population of exosomes is isolated based on the affinity to the immobilized antibody or binding portion thereof to perforin, TRAIL and/or granzyme B and/or Fas. In some alternatives, a population of exosomes is isolated, which comprises CD63 RNAs, and/or a desired microRNA. In some alternatives, a population of exosomes is isolated and/or characterized after isolation using affinity chromatography or immunological techniques, wherein said population of exosomes comprise markers or peptides such as small Rab family GTPases, annexins, flotillin, Alix, Tsg101, ESCRT complex, CD9, CD37, CD53, CD63, CD63A, CD81, CD82), Hsp70, Hsp90) and/or epithelial cell adhesion molecules (EpCam). As detailed above, in some alternatives, a first population of exosomes isolated from human placenta or a portion thereof by the methods described herein are contacted with a substrate having an immobilized antibody or binding portion thereof (e.g., a membrane, a resin, a bead, or a vessel having said immobilized antibody or binding portion thereof), wherein the immobilized antibody or binding portion thereof is specific for small Rab family GTPases, annexins, flotillin, Alix, Tsg101, ESCRT complex, CD9, CD37, CD53, CD63, CD63A, CD81, CD82), Hsp70, Hsp90) and/or epithelial cell adhesion molecules (EpCam) and a second population of exosomes from the first population of exosomes is isolated based on the affinity to the immobilized antibody or binding portion thereof to small Rab family GTPases, annexins, flotillin, Alix, Tsg101, ESCRT complex, CD9, CD37, CD53, CD63, CD63A, CD81, CD82), Hsp70, Hsp90) and/or epithelial cell adhesion molecules (EpCam). In other alternatives, a population of exosomes isolated from human placenta or a portion thereof by the methods described herein are contacted with an antibody or binding portion thereof specific for one or more of small Rab family GTPases, annexins, flotillin, Alix, Tsg101, ESCRT complex, CD9, CD37, CD53, CD63, CD63A, CD81, CD82, Hsp70, Hsp90 and/or epithelial cell adhesion molecules (EpCam) and the binding of the antibody or binding portion thereof is detected with a secondary binding agent having a detectable reagent, which binds to said antibody or binding portion thereof (e.g., utilizing an ELISA or blotting procedure) so as to confirm the presence of the small Rab family GTPases, annexins, flotillin, Alix, Tsg101, ESCRT complex, CD9, CD37, CD53, CD63, CD63A, CD81, CD82), Hsp70, Hsp90 and/or epithelial cell adhesion molecules (EpCam) in the isolated exosome population.

“Isolation” as described herein is a method for separating the exosomes from other materials. Isolation of exosomes may be performed by high centrifugal force in a centrifuge, utilization of commercially available kits (e.g. SeraMir Exosome RNA Purification kit (SBI system biosciences), Intact Exosome Purification and RNA Isolation (CombinationKit) Norgen BioTek Corp.), and the use of lectin affinity or affinity chromatography with binding agents (e.g., an antibody or binding portion thereof) specific for markers or peptides on the exosomes such as the markers or peptides mentioned above (e.g., binding agents specific for small Rab family GTPases, annexins, flotillin, Alix, Tsg101, ESCRT complex, CD9, CD37, CD53, CD63, CD63A, CD81, CD82), Hsp70, Hsp90, epithelial cell adhesion molecules (EpCam), perforin, TRAIL, granzyme B, Fas, one or more cancer markers such as: Fas ligand, CD24, EpCAM, EDIL3, fibronectin, Survivin, PCA3, TMPRSS2:ERG, Glypican-1, TGF-β1, MAGE 3/6, EGFR, EGFRvIII, CD9, CD147, CA-125, EpCam, and/or CD24, or one or more inflammatory or pathogenic markers such as: a viral, fungal, or a bacterial protein or peptide including but not limited to α-synuclein, HIV or HCV proteins, tau, beta-amyloid, TGF-beta, TNF-alpha, fetuin-A, and/or CD133).

“Placenta” as described herein is an organ in the uterus of pregnant eutherian mammals, nourishing and maintaining the fetus through the umbilical cord. As described herein, the placenta may be used as a bioreactor for obtaining exosomes. In some alternatives, a decellularized placenta may be used as a scaffold and bioreactor, which harbors an exogenous cell population (e.g., a cell population that has been seeded onto and cultured with the decellularized placenta) so as to obtain a population of exosomes from said cells, which are cell specific. Accordingly, in some alternatives, decellularized placenta is seeded with a regenerative cell population (e.g., a population of cells comprising stem cells and/or endothelial cells and/or progenitor cells) and said regenerative cell population is cultured on said decellularized placenta in a bioreactor and cell specific exosomes are isolated from said cultured cells using centrifugation, a commercially available exosome isolation kit, lectin affinity, and/or affinity chromatography using a binding agents (e.g., an antibody or binding portion thereof) specific for markers or peptides on the exosomes such as the markers or peptides mentioned above (e.g., binding agents specific for small Rab family GTPases, annexins, flotillin, Alix, Tsg101, ESCRT complex, CD9, CD37, CD53, CD63, CD63A, CD81, CD82), Hsp70, Hsp90, epithelial cell adhesion molecules (EpCam), perforin, TRAIL, granzyme B, Fas, one or more cancer markers such as: Fas ligand, CD24, EpCAM, EDIL3, fibronectin, Survivin, PCA3, TMPRSS2:ERG, Glypican-1, TGF-β1, MAGE 3/6, EGFR, EGFRvIII, CD9, CD147, CA-125, EpCam, and/or CD24, or one or more inflammatory or pathogenic markers such as: a viral, fungal, or a bacterial protein or peptide including but not limited to α-synuclein, HIV or HCV proteins, tau, beta-amyloid, TGF-beta, TNF-alpha, fetuin-A, and/or CD133).

“Ascites fluid” as described herein is excess fluid in the space between the membranes lining the abdomen and abdominal organs (the peritoneal cavity). Ascites fluid may be a source of exosomes.

“Plasma” as described herein is the liquid part of the blood and lymphatic fluid, which makes up about half of the volume of blood. Plasma is devoid of cells and, unlike serum, has not clotted. Blood plasma contains antibodies and other proteins. Plasma may be a source of exosomes.

Several methods of culturing cells so as to produce copious amounts exosomes are provided herein. Culture media used for recovering or isolating the exosomes may be provided with one or more nutrients, enzymes or chelators. Chelators may be used to facilitate release of the exosomes from the cultured cells. Without being limiting, chelators used in some of the methods may include a phosphonate, BAPTA tetrasodium salt, BAPTA/AM, Di-Notrophen TM reagent tetrasodium salt, EGTA/AM, pyridoxal isonicotinoyl hydrazine, N,N,N′,N′-tetrakis-(2 Pyridylmethyl)ethylenediamine, 6-Bromo-N′-(2-hydroxybenzylidene)-2-methylquinoline-4-carbohydrazide, 1,2-Bis(2-aminophenoxy)ethane-N,N,N′,N′-tetraacetic acid tetrakis(acetoxymethyl ester), (Ethylenedinitrilo)tetraacetic acid, (EDTA), Edathamil, Ethylenedinitrilotetraacetic acid, Ethylene glycol-bis(2-aminoethylether)-N,N,N,N′-tetraacetic acid, or Ethylene glycol-bis(β-aminoethyl ether)-N,N,N′,N′-tetraacetic acid (EGTA) or any combination thereof. The chelator may be provided in the media used to culture or isolate the exosomes at a concentration of 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM or 100 mM or at a concentration that is within a range defined by any two aforementioned concentrations. As shown herein, the presence of one or more chelators in the media unexpectedly enhanced recovery of exosomes from placenta cultured in a bioreactor. The media used to culture and/or recover the exosomes may also have a protease, which may further enhance the release of exosomes. In some alternatives, the protease provided in the media is trypsin, collagenase, chymotrypsin or carboxypeptidase. In some alternatives, the protease is provided in the media at a concentration of 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM or 100 mM or at a concentration that is within a range defined by any two of the aforementioned concentrations. One or more sugars may also be added to the media used to culture and/or recover the exosomes. In some alternatives, the sugar added to the media is glucose. It is contemplated that the presence of glucose in the media enhances the release of the exosomes. In some alternatives, the glucose is provided in the media at a concentration of 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM or 100 mM or at a concentration that is within a range defined by any two of the aforementioned concentrations. The media may also include growth factors, cytokines, or one or more drugs e.g., GM-CSF, serum and/or an AHR antagonist.

Methods of Collecting Exosomes from a Placenta or Portion Thereof

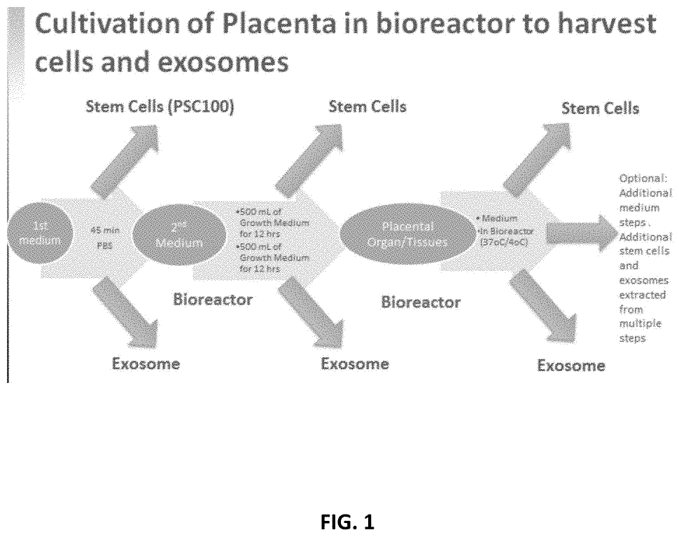

An exemplary method for recovery of exosomes from placenta is shown in FIG. 1. Sources for the exosome isolation may be from cord blood plasma: PRP, placenta perfusate (PS), placenta tissue cultivate (PTS), placenta organ cultivate (PO), or exogenous cells that may be placed in the placenta or portion thereof, when the placenta is used as a bioreactor for exosome generation. By one approach, placenta or portion thereof is collected (#200010323, collected Sep. 25, 2017). Placenta is contacted with a media or perfused with normal PSC-100 collection methods, collected as PS-1 (Sep. 26, 2017). The placenta or portion thereof is incubated in a hood for at least 4 hours. The placenta or portion thereof is contacted with media (RPMI media) or perfused with 500 mL RPMI base medium (1% antibiotics), collected as PS-2. The placenta or portion thereof is then incubated in a hood overnight and is covered. The placenta or portion thereof is contacted with or perfused with 750 mL saline solution and collected as PS-3. The samples were then shipped to a laboratory for analysis (Warren). PS1, PS2 and PS3 were analyzed by FACS at the same day after RBC lysis.

For the analysis, placenta tissue were cut into 1×1×1 cm size, placed in 100 mL of solution (all with 1% P&S) in T75 flasks (each about ⅛ of the placenta). Four solutions were assayed: A: DMEM medium; B: PBS; C: PBS+5 mM EDTA; D: PBS+0.025% Trypsin-EDTA. This was then allowed to incubate in 37° C. incubator overnight (0/N).

The supernatant was then harvested, passed through tissue filter and spun down at 400 g to harvest cells (pellet). The supernatant after the first centrifugation was then spun down for exosome isolation (3000 g spin soup>10,000 spin soup: 100,000 g pellet)

The cells collected were also used for FACS analysis. The cell samples were in several buffers (A=PTS1; B=PTS2; C=PTS-3, D=PTS4). Exosomes were recovered and were then assayed to identify the presence of an exosome marker confirming that the exosomes were obtained and isolated by the procedure.

Identification of a Population of Exosomes Isolated from the Placental Bioreactor Using ELISA and Protein Assays

Fractions of supernatant from the placental bioreactor were collected by the methods described above and the fractions were filtered. The supernatant was then subjected to centrifugation at 400 g×10 min to collect the cells. After the first centrifugation, a second centrifugation was performed at 3000 g×30 min to pellet cell debris. A third centrifugation was the performed at 10,000 g×1 hr to pellet micro vesicles. A fourth centrifugation was then performed at 100,000 g×1.5 hr to pellet exosomes. The centrifuge tube containing the pelleted exosomes was then placed upside-down on paper to drain residual liquid. The exosome pellet was then dissolved in an appropriate volume of sterile PBS (e.g. 2.0 mL) to dissolve pellet, and the solution containing the exosomes was then aliquoted in a sterile Eppendorf tube and frozen in a −20° C./−80° C. freezer. Exosomes were then assayed for the presence of an exosome-specific marker CD63A using an ELISA-63A and Protein Quantification Kit.

As shown, PRP, placenta perfusate and placenta tissue contain a population of exosomes that are CD63+ and can be efficiently isolated by ultracentrifiguation. For the exosome isolation, first the culture supernatant was filtered through a tissue filter and several centrifugations were performed as described above to obtain the exosomes, which were then frozen. For the ELISA detection of the exosomes, an anti-CD63 antibody was used. The sample was diluted 1:1 with exosome binding buffer (60 uL+60 uL) in the assay. CD63+ exosomes were efficiently isolated by this procedure.

### Characterization of Exosomes

Exosomes may contain protein, peptides, RNA, DNA and cytokines. Methods such as miRNA sequencing, surface protein analysis (MACSPlex Exosome Kit, Miltenyi), proteomic analysis, functional studies (enzyme assays in vitro wound healing assays (scratch assay), exosome-induced cell proliferation (human keratinocytes or fibroblast) (comparing to 5 known stimulants), exosome-induced collagen production (human keratinocyte or fibroblast): comparing to TGFb, includes serum and non-serum control, ELISA for pro-collagen 1 C peptide, exosome-induced inhibition of inflammatory cytokines: response cell types include human keratinocytes or human fibroblasts, and comparisons to lyophilized heat-killed bacterial or LPS) may be performed.

In some alternatives, isolated exosomes were concentrated with 100-Kda Vivaspin filter (Sartorius), washed once with PBS and approximately 40 uL was recovered. The concentrated population of exosomes was mixed with 10 uL of 5XRIPA lysis buffer containing 1×protease inhibitor cocktail (Roche) and vortexed, which was then followed by sonication at 20° C. for 5 min at a water sonicator (Ultrasonic Cleaner, JSP). After sonication, the tube was incubated on ice for 20 min with intermittent mixing. Next, the mixture was centrifuged at 10,000 g for 10 min at 4° C. The isolated clear lysate was transferred to a fresh tube. The protein amount was measured with BCA kit and 10 ug of protein was loaded per lane for Western blotting and an antibody is used for determination of a protein of interest.

In another alternative, exosome labeling and uptake by cells is examined (e.g. HEK293T). An aliquot of frozen eluted exosomes were resuspended in 1 mL of PBS and labeled using PKH26 Fluorescent cell linker Kits (Sigma-Aldrich). A 2×PNK26-dye solution (4 uL dye in 1 mL of Diluent C) was prepared and mixed with 1 mL of exosomal solution for a final dye concentration of 2×10e−6M. The samples was immediately mixed for 5 min and staining was stopped by adding 1% BSA to capture excel PKH26 dye. The labeled exosomes was transferred into a 100-Kda Vivaspin filter and spun at 4000 g then washed with PBS twice and approximately 50 uL of sample was recovered for analysis of exosome concentration using NTA prior to storage at −80 C. PBS was used as negative control for the labeling reaction. To perform the uptake studies, HEK293T cells were plated in 8-well chamber slide (1×10e4/vvell) using regular medium. After 24 hr, the slides was washed twice with PBS and incubated with DMEM-exo-free FBS (10%) for 24 hr. Following this, fresh DMEM media with 10% exo-free PBS (200 uL) each labeled exosome sample, corresponding to 2×10e9 exosomes, was added to each well and incubated for 1.5 hr in a cell culture incubator. After incubation, the slides was washed twice with PBS (500 ul) and fixed with 4% paraformaldehyde solution for 20 min at room temperature. The slides were washed twice with PBS (500 uL), dried, and mounted using a ProLong Gold Antifade Reagent with DAPI. The cells were visualized using an Axioskop microscope (Zeiss)

High Yield Isolation of Exosomes from Cultivated Postpartum Human Placenta

Postpartum human placentas obtained with full donor consent were perfused. Residual blood from the placenta was washed off with a large volume of sterile saline and then cultivated in a 5-L bioreactor with serum free culture medium supplemented with antibiotics and cultivated at 37° C. incubator (5% CO2) and alternated with rotating at refrigerated conditions for extended period unto to 4 days. Supernatant of the culture medium was processed by sequential centrifugation by 3000 g and 10,000 g to pellet tissue, cell and micro-vesicles. Exosomes were pelleted by 100,000 g ultra-centrifugation from the supernatant of 10,000 g centrifugation and dissolved with sterile PBS. The yield of exosome was quantified by BCA protein assay.

Supernatants from the placenta organ culture were processed as described in the methods to isolate exosomes. An ELISA assay using anti-CD63A antibodies demonstrated that the isolated exosomes contain the CD63A protein, a specific protein marker for exosomes. It is estimated one placenta cultured in one liter of medium generated approximately 40 mg of exosomes, or approximately 1×10¹³ CD63A positive exosome particles in 24 hours. Further characterization of these placenta-organ derived exosomes including expression of CD9, CD81, size and functional activities are performed.

In another set of experiments, postpartum human placentas obtained with full donor consent are perfused to isolate exosomes with media's having different concentrations of EDTA. Serum free culture medium supplemented with antibiotics and varying concentrations of EDTA (e.g., 5, 10, 20, 30, 40, 50, 60, 70, 80, 90, or 100 mM or within a range defined by any two of the aforementioned concentrations) are perfused into placenta through umbilical cord veins via peristaltic pump with a constant rate and cultivated another 24-48 hours under controlled conditions. Following this cultivation, 750 mL of physiologic medium containing the amount of EDTA employed is perfused at controlled rate. Exosomes are then isolated by sequential centrifugation and ultracentrifugation, confirmed by the CD63A ELISA assay, and quantified by the BCA protein assay, all described above. It will be shown that the concentration of EDTA in the media used to recover the exosomes impacts the amount of exosomes recovered from the placenta cultured in the bioreactor.

### Additional Alternatives

In some alternatives, a method of exosome isolation from a placenta or a portion thereof is provided. The method comprises a) contacting the placenta or a portion thereof with a first medium; b) obtaining a first fraction comprising exosomes from said placenta or portion thereof; c) contacting said placenta or portion thereof with a second medium; d) obtaining a second fraction comprising exosomes from said placenta or portion thereof, e) contacting said placenta or portion thereof with a third medium; f) obtaining a third fraction comprising exosomes from said placenta or portion thereof and, optionally, isolating the exosomes from said first, second, and/or third fractions. In some alternatives, the method further comprises multiple steps of contacting the placenta or portion thereof with an additional medium; and obtaining an additional fraction comprising exosomes from said placenta or portion thereof. These two steps may be repeated multiple times. Preferably, the placenta or portion thereof is cultured and/or maintained in a bioreactor. In some alternatives, the placenta or portion thereof comprises amniotic membrane. In some alternatives, the placenta or a portion thereof is a human placenta or a portion thereof. In some alternatives, the first, second, and/or third mediums are in contact with the placenta or portion thereof for at least 45 minutes, such as 45 minutes or 1, 2, 3, 4, 5, 6, 7, 8, 9, 10, 11 or 12 hours or any amount of time that is within a range defined by any two of the aforementioned time points. In some alternatives, the first, second, and/or third mediums are in contact with the placenta or portion thereof for at least 7, 14, 28, 35 or 42 days or any amount of time that is within a range defined by any two of the aforementioned time points. In some alternatives, the placenta or a portion thereof has been minced, ground, or treated with an enzyme such as collagenase and/or a protease.

In some alternatives, a placenta or a portion thereof is provided as a substantially flat or sheet-like scaffold material, which has been decellularized and, optionally, substantially dried. The decellularized placenta or a portion thereof is used as a scaffold to harbor exogenous cells such as homogeneous cell populations obtained from cell culture or primary isolation procedures (e.g., regenerative cells including stem cells, endothelial cells, and/or progenitor cells). The method further comprises passaging fluid or fluid comprising the cells to be seeded into the decellularized placenta or portion thereof. Once the cells are established, exosomes generated from the cells are recovered and isolated using the procedures described above. In some alternatives, the fluid comprising the cells to be seeded on the decellularized placenta or portion thereof is ascites fluid, blood or plasma. In some alternatives, the cells are from an organ. In some alternatives, the cells are from liver, kidney, lung or pancreas. In some alternatives, the cells are immune cells. In some alternatives, the cells are T-cells or B-cells.

In some alternatives, the first medium comprises Phosphate buffered saline (PBS). In some alternatives, the second medium comprises growth factors. In some alternatives, the third medium comprises a chelator. In some alternatives, the chelator is EDTA, EGTA, a phosphonate, BAPTA tetrasodium salt, BAPTA/AM, Di-Notrophen TM reagent tetrasodium salt, EGTA/AM, pyridoxal isonicotinoyl hydrazine, N,N,N′,N′-tetrakis-(2 Pyridylmethyl)ethylenediamine, 6-Bromo-N′-(2-hydroxybenzylidene)-2-methylquinoline-4-carbohydrazide, 1,2-Bis(2-aminophenoxy)ethane-N,N,N′,N′-tetraacetic acid tetrakis(acetoxymethyl ester), (Ethylenedinitrilo)tetraacetic acid, EDTA, Edathamil, Ethylenedinitrilotetraacetic acid, Ethylene glycol-bis(2-aminoethylether)-N,N,N′,N′-tetraacetic acid, or Ethylene glycol-bis(β-aminoethyl ether)-N,N,N′,N′-tetraacetic acid tetrasodium salt or any combination thereof. In some alternatives, the chelator is EDTA or EGTA or a combination thereof. In some alternatives, the chelator is provided in the third medium at a concentration of 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM or 100 mM or at a concentration that is within a range defined by any two aforementioned concentrations. In some alternatives, the concentration of EDTA in the third medium is provided at a concentration of 1 mM, 2 mM, 3 mM, 4 mM, 5 mM, 6 mM, 7 mM, 8 mM, 9 mM 10 mM, 20 mM, 30 mM, 40 mM, 50 mM, 60 mM, 70 mM, 80 mM, 90 mM or 100 mM or at a concentration that is within a range defined by any two aforementioned concentrations.

In some alternatives, the third medium comprises a protease. In some alternatives, the protease is a trypsin, collagenase, chymotrypsin or carboxypeptidase or a mixture thereof. In some alternatives, the protease is trypsin. In some alternatives, the protease is provided in the third medium at a concentration of 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM or 100 mM or at a concentration that is within a range defined by any two of the aforementioned concentrations.

In some alternatives, the method further comprises contacting the placenta or portion thereof with an additional plurality of mediums, wherein the contacting results in obtaining multiple fractions comprising exosomes. In some alternatives, the first, second, third or additional mediums comprise glucose. In some alternatives, the first, second, third or additional mediums comprise GM-CSF. In some alternatives, the first, second, third or additional mediums comprise serum. In some alternatives, the first, second, third or additional mediums comprise DMEM. In some alternatives, the first, second, third or additional medium comprises an AHR antagonist. In some alternatives, the AHR antagonist is SR1. In some alternatives, the SR1 is at a concentration of 1 nM, 10 nM, 100 nM, 200 nM, 300 nM, 400 nM, 500 nM, 600 nM, 700 nM, 800 nM, 900 nM or 1 mM or any other concentration within a range defined by any two aforementioned values.

In some alternatives, the first medium is in contact with the placenta or portion thereof while maintaining a temperature of 0° C., 5° C., 10° C., 15° C., 20° C., 25° C., 30° C., 35° C. or 40° C. or a temperature that is within a range defined by any two of the aforementioned temperatures. In some alternatives, the second medium is in contact with the placenta or portion thereof while maintaining a temperature of 0° C., 5° C., 10° C., 15° C., 20° C., 25° C., 30° C., 35° C. or 40° C. or a temperature that is within a range defined by any two of the aforementioned temperatures. In some alternatives, the third medium is in contact with the placenta or portion thereof while maintaining a temperature of 0° C., 5° C., 10° C., 15° C., 20° C., 25° C., 30° C., 35° C. or 40° C. or a temperature that is within a range defined by any two of the aforementioned values. In some alternatives, the additional plurality of mediums is in contact with the placenta or portion thereof while maintaining a temperature of 0° C., 5° C., 10° C., 15° C., 20° C., 25° C., 30° C., 35° C. or 40° C. or a temperature that is within a range defined by any two of the aforementioned values.

In some alternatives, the first, second or third media or additional plurality of mediums comprise antibiotics.

In some alternatives, the exosomes are isolated from said first, second, and/or third fractions or multiple fractions by a method comprising:
(a) passing the first, second and/or third fractions or multiple fractions through a tissue filter;
(b) performing a first centrifugation of the filtrate collected in (a) to generate a cell pellet and a first supernatant;
(c) performing a second centrifugation on the first supernatant to generate a second supernatant; and
(d) performing a third centrifugation on the second supernatant to generate an exosome pellet; and, optionally,
(e) resuspending the exosomes in a solution.

In some alternatives, the population of isolated exosomes comprise exosomes having CD63, CD63-A, perforin, Fas, TRAIL or granzyme B Bor a combination thereof. In some alternatives, the population of isolated exosomes comprise exosomes that comprise a signaling molecule. In some alternatives, the population of isolated exosomes comprise exosomes that comprise cytokines, mRNA or miRNA.

In some alternatives, the method further comprises isolating exosomes by affinity chromatography, wherein affinity chromatography is selective for the removal of exosomes comprising viral antigens, viral proteins, bacterial antigens, or bacterial protein fungal antigens or fungal proteins.

In some alternatives, the method further comprises isolating exosomes by an alternative or additional affinity chromatography step, wherein the alternative or additional affinity chromatography step is selective for the removal of exosomes comprising inflammatory proteins. In some alternatives, the method further comprises enriching a population of exosomes comprising anti-inflammatory biomolecules.

In some alternatives, exosomes generated by any one of the embodiments herein are provided. In some alternatives, the exosomes are from ascites fluid, blood or plasma. In some alternatives, the exosomes are from cells from an organ. In some alternatives, the exosomes are from immune cells. In some alternatives, the exosomes are from T-cells or B-cells.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., a system having at least one of A, B, and C″ would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., a system having at least one of A, B, or C″ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

### 6. EXAMPLES

### 6.1. Example 1: Cultivation of Human Placenta

Human placenta are received and washed with sterile PBS or saline solution to remove blood. The placenta is then cultivated in vessels as a whole organ in a large container with volume of 500 mL or 1000 mL of DMEM culture media supplemented with antibiotics and 2 mM EDTA. In a different alternative, the placenta can be cut into different sizes and placed in the culture container. The cultivation is at 37° C. in cell culture incubator with 5% CO2. The cultivation time is 4 hour to 8 hours and the supernatant of the culture is used for isolation of exosomes. New media is added at each harvest time point (e.g., every 8 hours or every 12 hours) and the placenta organ and tissue is cultured for up to at least 5 days.

### 6.2. Example 2: Isolation and Purification of Placenta Exosomes

The supernatant of the culture is centrifuged at 3,000 g for 30 minutes to pellet the cell and tissue debris. The supernatant is then centrifuged at 10,000 g for 1 hour and the pellet (small cell debris and organelles) is discarded. The supernatant is then centrifuged at 100,000 g for 2 hours. The resulted pellet is exosomes. The exosomes pellet can be further purified by the following method: resuspended with different volume of sterile PBS and centrifuged again at 100,000 for 2 hours and the final pellet is then resuspended with sterile PBS. The resuspended exosome is filtered through a syringe filter (0.2 um), aliquoted at −80oC at different volumes from 300 uL to 1 mL.

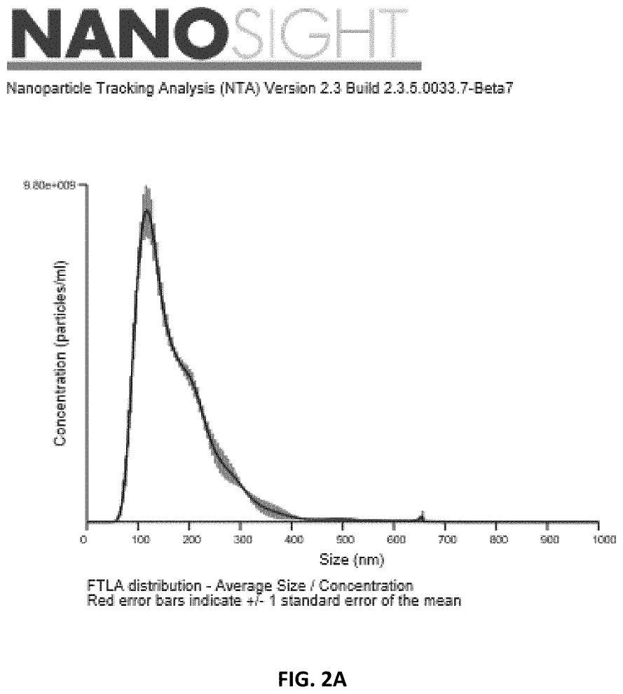

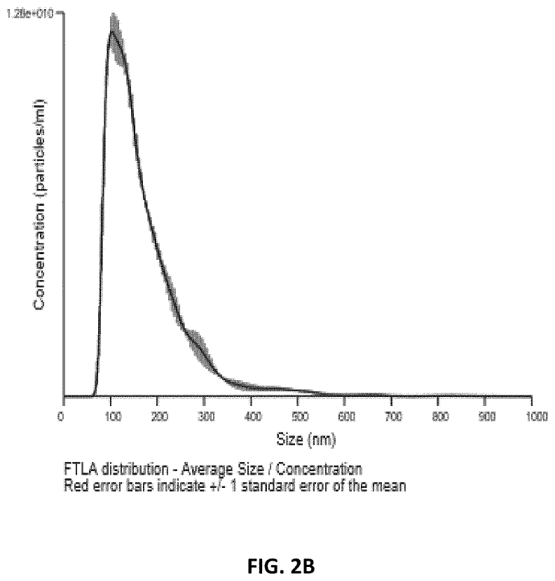

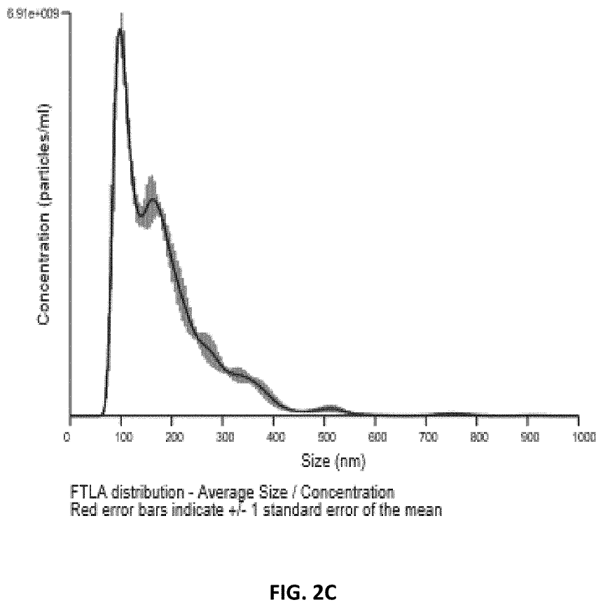

Placental exosomes are characterized by size. Size distribution is analyzed by a nanoparticle tracking assay. Three representative samples of pExo were measured with their size using NanoSight. Each isolate has a mean size of 117, 101, and 96 respectively, consistent with the reported size of exosomes. Results are shown in FIG. 2A-FIG. 2C.

### 6.3. Example 3: Markers of pExos by FACS Analysis

Protein markers of pExo were analyzed with MACSPlex Exosome Kit (Miltenyi Biotec, Cat #130-108-813) following the protocol provided by the kit. Briefly, the 120 uL of pExo isolates were incubated with 15 uL of exosome capture beads overnight at room temperature overnight. After washing once with 1 mL wash solution, the exosome were incubated with exosome detection reagents CD9, CD63 and CD81 cocktail and incubated for additional 1 hrs. After two washes, the samples were analyzed with FACS (BD Canto 10). There are total 37 proteins markers included in this kit (Table 1) excluding mIgG1 and REA control.

TABLE 1
List of protein markers used to detect pExo in MACSPlex Exosome Kit

| No. | Antibody | Isotype |
|---|---|---|
| 22 | CD3 | mIgG2a |
| 23 | CD4 | mIgG2a |
| 24 | CD19 | mIgG1 |
| 32 | CD8 | mIgG2a |
| 33 | HLA-DRDPDQ | REA |
| 34 | CD56 | REA |
| 35 | CD105 | mIgG1 |
| 42 | CD2 | mIgG2b |
| 43 | CD1c | mIgG2a |
| 44 | CD25 | mIgG1 |
| 45 | CD49e | mIgG2b |
| 46 | ROR1 | mIgG1κ |
| 52 | CD209 | mIgG1 |
| 53 | CD9 | mIgG1 |
| 54 | SSEA-4 | REA |
| 55 | HLA-ABC | REA |
| 56 | CD63 | mIgG1κ |
| 57 | CD40 | mIgG1κ |
| 63 | CD62P | REA |
| 64 | CD11c | mIgG2b |
| 65 | CD81 | REA |
| 66 | MCSP | mIgG1 |
| 67 | CD146 | mIgG1 |
| 68 | CD41b | REA |
| 74 | CD42a | REA |
| 75 | CD24 | mIgG1 |
| 76 | CD86 | mIgG1 |
| 77 | CD44 | mIgG1 |
| 78 | CD326 | mIgG1 |
| 79 | CD133/1 | mIgG1κ |
| 85 | CD29 | mIgG1κ |
| 86 | CD69 | mIgG1κ |
| 87 | CD142 | mIgG1κ |
| 88 | CD45 | mIgG2a |
| 89 | CD31 | mIgG1 |
| 96 | REA Control | REA |
| 97 | CD20 | mIgG1 |
| 98 | CD14 | mIgG2a |
| 99 | mIgG1 control | mIgG1 |

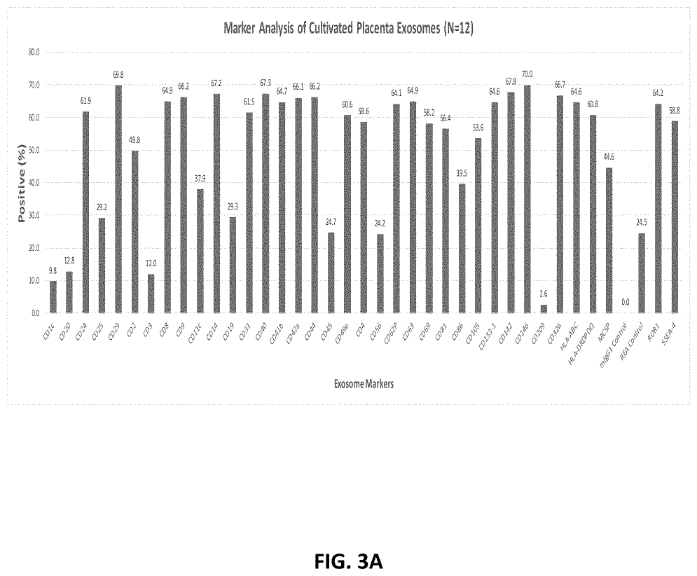

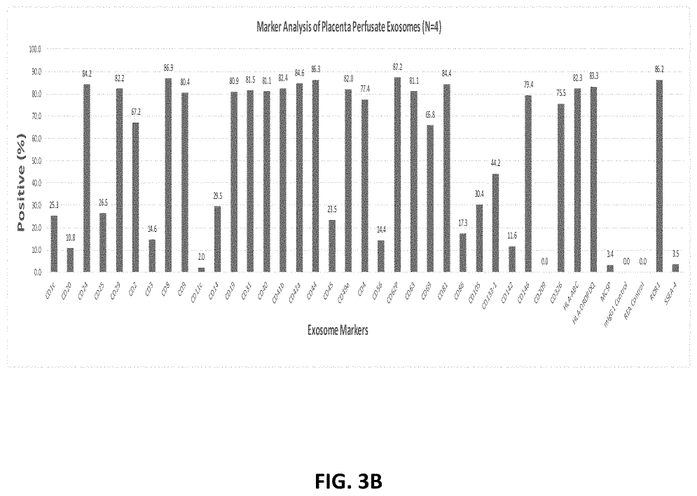

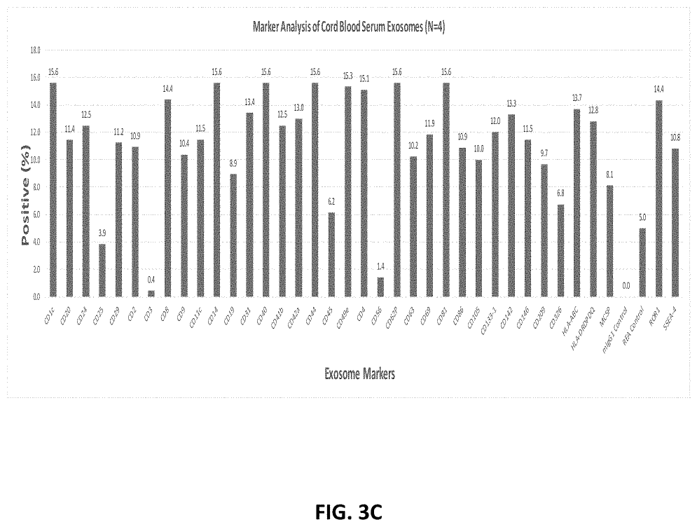

pExo samples were identified to be highly positive for the following protein markers including CD1c, CD9, CD20, CD24, CD25, CD29, CD2, CD3, CD8, CD9, CD11c, CD14, CD19, CD31, CD10, CD41b, CD42a, CD44, CD45, CD19c, CD4, CD15, CD19c, CD4, CD56, CD62P, CD83, CD69, CD81, CD86, CD105, CD133-1, CD142, CD148, HLA-ABC, HLA-DRDPDQ, MSCP, ROR1, SSEA-4. pExo has very low level (2.6%) in CD209. Human placenta perfusate, which is obtained by perfuse the vasculature of placenta with saline solution without cultivation with medium and cell culture incubator, was also used to isolate exosomes and analyzed by the same methods for marker protein expression. The perfusate derived exosomes also express high levels of most of the markers found in pExo, but it has significantly lower CD11c (2.0%), MCSP (3.4%) and SSEA-4 (3.50/6) comparing with pExos. pExo also has significantly higher levels of CD142 and CD81 comparing with placenta perfusate exosomes. Umbilical cord blood serum was also used to isolate exosomes and analyzed by the same methods for parker protei expression. Cord blood serum derived exosomes are also positive in most of the protein markers, but in general shows lower levels of each these marker protein expressions. Specifically, comparing with pExo, cord blood serum exosome has lower levels of CD56 (1.4%), CD3 (0.3%) and CD25 (3.9%). SSEA-4 and MSCP protein expression in cord blood serum is significantly lower than pExo but higher than placenta perfusate exosomes. Cord blood serum exosomes also has higher levels of MSCP protein comparing with pExo. These data indicate that cultivated placenta tissues can generate a unique exosome population comparing with non-cultured placenta and cord blood serum. Results for pExo samples, compared to cord blood serum derived exosomes and placenta perfusate exosomes are shown in FIG. 3A-FIG. 3C and Table 2.

TABLE 2
Protein Markers of Average Expression (%) on
Exosomes from Three Different Sources

| | Cultivated | | |
|---|---|---|---|
| | Placenta | Placenta Perfusate | Cord Blood Serum |
| Markers | (N = 12) | (N = 4) | (N = 4) |
| CD1c | 9.80% | 25.30% | 15.60% |
| CD20 | 12.80% | 10.80% | 11.40% |
| CD24 | 61.90% | 84.20% | 12.50% |
| CD25 | 29.20% | 26.50% | 3.90% |
| CD29 | 69.80% | 82.20% | 11.20% |
| CD2 | 49.80% | 67.20% | 10.90% |
| CD3 | 12.00% | 14.60% | 0.40% |
| CD8 | 64.90% | 86.90% | 14.40% |
| CD9 | 66.20% | 80.40% | 10.40% |
| CD11c | 37.90% | 2.00% | 11.50% |
| CD14 | 67.20% | 29.50% | 15.60% |
| CD19 | 29.30% | 80.90% | 8.90% |
| CD31 | 61.50% | 81.50% | 13.40% |
| CD40 | 67.30% | 81.10% | 15.60% |
| CD41b | 64.70% | 82.40% | 12.50% |
| CD42a | 66.10% | 84.60% | 13.00% |
| CD44 | 66.20% | 86.30% | 15.60% |
| CD45 | 24.70% | 23.50% | 6.20% |
| CD49e | 60.60% | 82.00% | 15.30% |
| CD4 | 58.60% | 77.40% | 15.10% |
| CD56 | 24.20% | 14.40% | 1.40% |
| CD62P | 64.10% | 87.20% | 15.60% |
| CD63 | 64.90% | 81.10% | 10.20% |
| CD69 | 58.20% | 65.80% | 11.90% |
| CD81 | 56.40% | 84.40% | 15.60% |
| CD86 | 39.50% | 17.30% | 10.90% |
| CD105 | 53.60% | 30.40% | 10.00% |
| CD133-1 | 64.60% | 44.20% | 12.00% |
| CD142 | 67.80% | 11.60% | 13.30% |
| CD146 | 70.00% | 79.40% | 11.50% |
| CD209 | 2.60% | 0% | 9.70% |
| CD326 | 66.70% | 75.50% | 6.80% |
| HLA-ABC | 64.60% | 82.30% | 13.70% |
| HLA- | 60.80% | 83.30% | 12.80% |
| DRDPDQ | | | |
| MCSP | 44.60% | 3.40% | 8.10% |
| ROR1 | 64.20% | 86.20% | 14.40% |
| SSEA-4 | 58.80% | 3.50% | 10.80% |

### 6.4. Example 4: Cytokines and Growth Factors of pExo Samples

pExo samples were analyzed for their contents of cytokines with MiltiPlex Luminex kit that includes 41 different cytokines. The following tables show the data of cytokines detected on 15 different pExo preparations. The data shows that pExo contains significant level of cytokines (mean >50 pg/mL) including FGF2, G-CSF, Fractalkine, GDGF-AA/BB, GRO, IL-IRA, IL-8, VEGF, and RANTES. pExo also contains detectable levels of cytokines (5 pg/mL to 49 pg/mL) of other cytokines including EGF, Flt-3L, IFNa3, MCP-3, PDGF-AA, IL-15, sCD40L, IL6, IP-10, MCP-1, MIP-alpha, MIP-lbeta, and TNF-alpha.

TABLE 3
Cytokines detected in pExo preparations

| | | | | | | | GM- | | | | | | | IL- |
|---|---|---|---|---|---|---|---|---|---|---|---|---|---|---|
| Sample ID | EGF | FGF-2 | Eotaxin | TGF-a | G-CSF | Flt-3L | CSF | Fractalkine | IFNa2 | IFNg | GRO | IL-10 | MCP-3 | 12P40 |
| (Table 1-1) | pg/ml | pg/ml | pg/ml | pg/ml | pg/ml | pg/ml | pg/ml | pg/ml | pg/ml | pg/ml | pg/ml | pg/ml | pg/ml | pg/ml |
| 3074-E1 | 2.79 | 17.11 | 3.77 | <0.55↓ | 249.56 | 1.57 | 0.49 | 40.25 | 7.1 | 0.61 | 40.44 | 0.59 | 5.87 | <0.74↓ |
| 3315-E1 | 4.41 | 290.32 | 8.47 | <0.55↓ | 64.49 | 6.83 | 1.12 | 83.56 | 11.3 | 1.2 | 108.91 | <0.57↓ | 4.32 | <0.74↓ |
| 941-E1 | 1.59 | 17.11 | <3.20↓ | <0.55↓ | 96.52 | <0.62↓ | <0.42↓ | 17.66 | 2.22 | 0.87 | 6.6 | 0.7 | 0.85 | <0.74↓ |
| 941-E2 | 1.59 | 12.33 | <3.20↓ | <0.55↓ | 141.85 | <0.62↓ | 0.45 | 22.66 | 5.19 | 1.01 | 6.6 | 0.62 | 1.59 | <0.74↓ |
| 988-E1 | 4.83 | 56.94 | 3.25 | 0.68 | 441.69 | 3.74 | 1.9 | 83.56 | 7.83 | 1.54 | 36.15 | 1.21 | 5.57 | <0.74↓ |
| 595-E2 | 12.76 | 120.53 | 11.42 | 2.03 | 267.84 | 5.42 | 2.2 | 227.72 | 13.81 | 1.93 | 102.16 | 1.63 | 4.32 | 2.66 |
| 595-E3 | 5 | 30.09 | 7.45 | <0.55↓ | 247.34 | 8.21 | 1.81 | 110.13 | 28.61 | 4.22 | 17.13 | 1.11 | <0.38↓ | 2.73 |
| 366-E2 | 6.18 | 359.37 | 6.56 | 1.27 | 343.71 | 12.73 | 1.71 | 197.68 | 7.35 | 1.46 | 103 | 2.33 | 9.97 | 1.96 |
| 405-E2 | 9.78 | 318.88 | 8.72 | 1.64 | 148.99 | 13.34 | 1.74 | 338.31 | 9.06 | 0.61 | 114.73 | 1.98 | 9.46 | 1.28 |
| 405-E3 | 7.91 | 226.62 | 6.29 | 0.84 | 179.5 | 4.95 | 1.53 | 225.33 | 7.47 | 0.48 | 96.86 | 1.21 | 6.75 | 1.73 |
| 352-E1 | 6.18 | 508.7 | 7.1 | 0.92 | 48.57 | 22.98 | 1.78 | 385.31 | 14.81 | 1.91 | 139.65 | 1.86 | 11.19 | 4.13 |
| 352-E2 | 5.16 | 483.27 | 6.29 | 0.78 | 72.77 | 15.12 | 1.38 | 251.86 | 10.68 | 1.31 | 109.76 | 1.14 | 5.57 | 2.21 |
| 789-E1 | 13.48 | 20.08 | 7.45 | 2.29 | 118.38 | <0.62↓ | 0.98 | 123.46 | 5.19 | <0.46↓ | 38.51 | 1.35 | 3 | 1.5 |
| 789-E2 | 5.72 | 24.95 | 5.83 | <0.55↓ | 159.06 | 1.1 | 1.56 | 61.1 | 4.16 | 0.94 | 24.96 | 0.88 | 5.87 | <0.74↓ |
| 313-E3 | 3.72 | 27.58 | 4.97 | <0.55↓ | 57.57 | <0.62↓ | 0.7 | 77.5 | 20.54 | 1.82 | 7.44 | 0.85 | <0.38↓ | <0.74↓ |
| | | | | | | | GM- | | | | | | | IL- |
| | EGF | FGF-2 | Eotaxin | TGF-a | G-CSF | Flt-3L | CSF | Fractalkine | IFNa2 | IFNg | GRO | IL-10 | MCP-3 | 12P40 |
| Mean | 6.07 | 167.59 | 6.74 | 1.31 | 175.86 | 8.73 | 1.38 | 149.74 | 10.35 | 1.42 | 63.53 | 1.25 | 5.72 | 2.28 |
| SD | 3.6 | 181.0 | 2.1 | 0.6 | 114.3 | 6.7 | 0.5 | 115.0 | 6.9 | 0.9 | 47.8 | 0.5 | 3.1 | 0.9 |

| Sample ID | MDC | IL-12P70 | PDGF-AA | IL-13 | PDGF-AB/BB | IL-15 | sCD40L | IL-17A | IL-1RA | IL-1a | IL-9 | IL-1b | IL-2 | IL-3 |
|---|---|---|---|---|---|---|---|---|---|---|---|---|---|---|
| (Table 1-2) | pg/ml | pg/ml | pg/ml | pg/ml | pg/ml | pg/ml | pg/ml | pg/ml | pg/ml | pg/ml | pg/ml | pg/ml | pg/ml | pg/ml |
| 3074-E1 | 8.33 | <0.71↓ | 3.95 | 1.3 | 203.83 | 2.19 | 0.81 | 0.41 | 10.87 | 0.77 | 0.87 | 0.48 | <0.42↓ | <0.31↓ |
| 3315-E1 | <7.64↓ | 1.12 | 14.15 | 2.02 | 314.97 | 8.37 | 0.74 | 0.39 | 124.57 | 0.53 | 1.29 | 0.85 | <0.42↓ | <0.31↓ |
| 941-E1 | <7.64↓ | <0.71↓ | 1.41 | 1.01 | 35.31 | 0.95 | 1.5 | <0.36↓ | 9.47 | 0.39 | 0.36 | 1.23 | <0.42↓ | <0.31↓ |
| 941-E2 | <7.64↓ | <0.71↓ | 3.59 | 0.97 | 93.37 | 1.2 | 0.81 | 0.46 | 3.48 | 0.69 | 0.62 | 7 | <0.42↓ | <0.31↓ |
| 988-E1 | <7.64↓ | 0.94 | 8.48 | 1.59 | 127 | 3.76 | 3.94 | 0.64 | 53.63 | 2.02 | 0.86 | 0.79 | <0.42↓ | <0.31↓ |
| 595-E2 | 8.57 | 3.07 | 21.5 | 4.25 | 506.7 | 4.66 | 25.66 | 0.95 | 92.19 | 2.7 | 1.84 | 8.2 | <0.42↓ | <0.31↓ |
| 595-E3 | 11.62 | 1.65 | 12.6 | 3.9 | 317.14 | 3.72 | 2.05 | 0.98 | 18.86 | 2.09 | 2.92 | 3.37 | 0.53 | 0.51 |
| 366-E2 | 19.46 | 1.65 | 23.19 | 1.49 | 439.81 | 8.44 | 22.25 | 0.9 | 110.52 | 2.55 | 0.99 | 1.39 | <0.42↓ | 0.37 |
| 405-E2 | 45.61 | 3.2 | 26.94 | 1.49 | 510.45 | 12.35 | 23.9 | 0.5 | 116.59 | 1.6 | 1.08 | 1.29 | <0.42↓ | <0.31↓ |
| 405-E3 | 24.28 | 1.16 | 18.87 | 1.28 | 335.8 | 10.21 | 10.81 | <0.36↓ | 90.53 | 1.12 | 0.84 | 1.68 | <0.42↓ | <0.31↓ |
| 352-E1 | 27.1 | 3.2 | 33.76 | 2.04 | 492.97 | 33.13 | 18.13 | 1.01 | 169.21 | 2.48 | 1.49 | 1.5 | 0.45 | <0.31↓ |
| 352-E2 | 14.83 | 2.14 | 28.14 | 1.21 | 442.07 | 23.78 | 11.72 | 0.98 | 107.61 | 1.87 | 1.18 | 1.38 | <0.42↓ | <0.31↓ |
| 789-E1 | <7.64↓ | 1.86 | 7.02 | 1.61 | 192.21 | 2.19 | 10.47 | 0.77 | 91.16 | 1.71 | 0.78 | 0.39 | <0.42↓ | <0.31↓ |
| 789-E2 | 9.75 | 1.97 | 9.3 | 1.03 | 210.26 | 1.8 | 0.94 | 0.64 | 18.86 | 1.25 | 0.91 | 0.64 | <0.42↓ | <0.31↓ |
| 313-E3 | <7.64↓ | 0.94 | 5.01 | 2.89 | 167.09 | 0.95 | <0.56↓ | 0.85 | <3.20↓ | 1.05 | 2.93 | 0.35 | <0.42↓ | 0.42 |
| | MDC | IL-12P70 | PDGF-AA | IL-13 | PDGF-AB/BB | IL-15 | sCD40L | IL-17A | IL-1RA | IL-1a | IL-9 | IL-1b | IL-2 | IL-3 |
| Mean | 18.84 | 1.91 | 14.53 | 1.87 | 292.60 | 7.85 | 9.55 | 0.73 | 72.69 | 1.52 | 1.26 | 2.02 | 0.49 | 0.43 |
| SD | 12.2 | 0.8 | 10.2 | 1.0 | 159.1 | 9.3 | 9.5 | 0.2 | 52.9 | 0.8 | 0.8 | 2.4 | 0.1 | 0.1 |

| Sample ID | IL-4 | IL-5 | IL-6 | IL-7 | IL-8 | IP-10 | MCP-1 | MIP-1a | MIP-1b | RANTES | TNFa | TNFb | VEGF |
|---|---|---|---|---|---|---|---|---|---|---|---|---|---|
| (Table 1-3) | pg/ml | pg/ml | pg/ml | pg/ml | pg/ml | pg/ml | pg/ml | pg/ml | pg/ml | pg/ml | pg/ml | pg/ml | pg/ml |
| 3074-E1 | <3.20↓ | <0.21↓ | 2.92 | 2.9 | 72.66 | 7.5 | 17.08 | 2.24 | 1.51 | 143.77 | 5.64 | 0.41 | 21.6 |
| 3315-E1 | <3.20↓ | <0.21↓ | 6.3 | 6.2 | 215.72 | 27.63 | 85.9 | 11.98 | 8.27 | 292.91 | 2.1 | 0.44 | 56.06 |
| 941-E1 | <3.20↓ | 0.27 | 1.15 | 1.45 | 6.08 | <1.30↓ | 1.67 | <1.31↓ | <0.33↓ | 48.16 | 2.67 | 0.49 | 39.7 |
| 941-E2 | <3.20↓ | <0.21↓ | 1.46 | 5.34 | 6.6 | <1.30↓ | 1.53 | 1.48 | 0.89 | 30.32 | 16.58 | 0.38 | 43.8 |
| 988-E1 | <3.20↓ | 0.27 | 9.07 | 3.6 | 58.25 | 47.16 | 20.48 | 2.95 | 2.99 | 396.33 | 25.8 | 0.59 | 59.12 |
| 595-E2 | <3.20↓ | 0.22 | 20.55 | 10.12 | 192.31 | 14.05 | 63.62 | 13.25 | 3.74 | 4482 | 23.97 | 0.41 | 51.98 |
| 595-E3 | <3.20↓ | 1.39 | 10.06 | 6.49 | 60.01 | 6.75 | 11.42 | 6.76 | 1.51 | 265.85 | 16.15 | 0.58 | 106.17 |
| 366-E2 | 5.54 | 0.47 | 15.93 | 4.55 | 103.91 | 101.77 | 71.51 | 21.83 | 11.8 | 2413 | 5.41 | 1.73 | 64.19 |
| 405-E2 | 4.01 | 0.38 | 17.02 | 5 | 105.05 | 92.1 | 99.23 | 28.81 | 16.62 | 2463 | 6.35 | 0.97 | 54.71 |
| 405-E3 | <3.20↓ | 0.32 | 13.3 | 3.6 | 159.18 | 53.34 | 59.98 | 27.54 | 17.63 | 1655 | 5.82 | 0.73 | 44.31 |
| 352-E1 | 6.08 | 0.45 | 24.21 | 7.24 | 167.95 | 156.45 | 138.26 | 9.99 | 8.19 | 3000 | 3.29 | 1.12 | 67.45 |
| 352-E2 | <3.20↓ | 0.38 | 18.92 | 5.4 | 198.95 | 89.91 | 103.45 | 12.06 | 6.69 | 2415 | 2.96 | 0.76 | 62.53 |
| 789-E1 | <3.20↓ | 0.35 | 2.62 | 3.01 | 17.58 | 5.64 | 8.44 | 1.82 | 0.85 | 659.52 | 7.28 | 0.55 | 24.19 |
| 789-E2 | <3.20↓ | 0.27 | 5.69 | 2.85 | 84.19 | 4.65 | 8.74 | 3.7 | 1.04 | 417.14 | 5.5 | 0.52 | 22.25 |
| 313-E3 | <3.20↓ | 0.61 | 1.11 | 10.52 | 8.32 | 3.67 | 4.2 | 3.33 | 0.53 | 189.04 | 3.83 | 0.52 | 60.41 |
| | IL-4 | IL-5 | IL-6 | IL-7 | IL-8 | IP-10 | MCP-1 | MIP-1a | MIP-1b | RANTES | TNFa | TNFb | VEGF |
| Mean | 5.21 | 0.45 | 10.02 | 5.22 | 97.12 | 46.97 | 46.37 | 10.55 | 5.88 | 1256.74 | 8.89 | 0.68 | 51.90 |
| SD | 1.1 | 0.3 | 7.8 | 2.6 | 74.1 | 49.1 | 45.2 | 9.4 | 5.9 | 1380.0 | 7.8 | 0.4 | 21.4 |

pExo (11 samples) were also analyzed for the presence of soluble cytokine receptors by Multiplex Luminex analysis. The data are shown in the following table. The data shows that pExo contains high levels (>100 pg/mL) of sEFGR, sgp-130, sIL-1R1, sTNFR1, sTNFRII, sVEGRR1, sVEGFR1, sVEGFR3 and sCD30, sIL-2Ra, sIL-6R, sRAGE are also detected in some samples (>10 ng/mL). Data shown as < are not detected and are regarded as negative.

TABLE 4
Soluble cytokine receptors in placenta exosomes

| | pExo | sCD30 | sEGFR | sgp-130 | sIL1-RI | sIL-1RII | sIL-2Ra | sIL-4R | sIL-6R |
|---|---|---|---|---|---|---|---|---|---|
| Location | Samples | pg/ml | pg/ml | pg/ml | pg/ml | pg/ml | pg/ml | pg/ml | pg/ml |
| 1E3 | 988E1 | 11.76 | 9626 | 326.15 | <12.67↓ | <35.04↓ | 8.5 | <59.19↓ | 10.54 |
| 1H3 | 595E1 | <6.77↓ | 3535 | 209.91 | <12.67↓ | <35.04↓ | <5.97↓ | <59.19↓ | 9.78 |
| 1C4 | 941E2 | 11.41 | 57601 | 132.85 | <12.67↓ | <35.04↓ | 6.57 | <59.19↓ | 2.85 |
| 1F4 | 941E1 | 8.03 | >1003047↑ | 83.08 | <12.67↓ | <35.04↓ | <5.97↓ | <59.19↓ | 3.65 |
| 1A5 | 405E1 | 12.84 | 5863 | 2316 | <12.67↓ | 206.01 | 11.6 | <59.19↓ | 197.58 |
| 1D5 | 366E1 | 9.34 | 10444 | 2806 | <12.67↓ | 250.03 | 21.23 | <59.19↓ | 232.91 |
| 1G5 | 354E2 | 19.12 | 10627 | 4461 | <12.67↓ | 327.31 | 17.59 | <59.19↓ | 172.5 |
| 1B6 | 352E1 | 14.68 | 7824 | 4108 | <12.67↓ | 474.46 | 16.59 | <59.19↓ | 183.25 |
| 1E6 | 789E1 | <6.77↓ | 25357 | 174.96 | <12.67↓ | <35.04↓ | <5.97↓ | <59.19↓ | 7.18 |
| 1H6 | 789E2 | <6.77↓ | 2499 | 206.92 | <12.67↓ | <35.04↓ | <5.97↓ | <59.19↓ | 6.55 |
| 1C7 | 789E3 | <6.77↓ | 2149 | 197.21 | <12.67↓ | <35.04↓ | <5.97↓ | <59.19↓ | 4.05 |
| | Mean | 12.45 | 13552.50 | 1365.64 | NA | 314.45 | 13.68 | NA | 75.53 |
| | SD | 3.66 | 16858.13 | 1725.87 | NA | 117.87 | 5.70 | NA | 97.06 |
| 1A3 | QC1 | 465.86 | 2089 | 412.75 | 408.59 | 1948 | 420.27 | 200.54 | 138.73 |
| 1C3 | QC2 | 4219 | 18434 | 4012 | 4060 | 17200 | 4021 | 2290 | 1996 |

| | pExo | sRAGE | sTNFRI | sTNFRII | sVEGFR1 | sVEGFR2 | sVEGFR3 |
|---|---|---|---|---|---|---|---|
| Location | Samples | pg/ml | pg/ml | pg/ml | pg/ml | pg/ml | pg/ml |
| 1E3 | 988E1 | 29.49 | 90.11 | 14.82 | 6556 | 75.84 | 462.01 |
| 1H3 | 595E1 | 8.23 | 29.88 | <12.55↓ | 2959 | <70.59↓ | 47.7 |
| 1C4 | 941E2 | 11.2 | 34.07 | 19.66 | 4929 | <70.59↓ | 119.96 |
| 1F4 | 941E1 | 7.57 | 25.57 | <12.55↓ | 803.42 | <70.59↓ | 56.61 |
| 1A5 | 405E1 | 17.06 | 253.83 | 365.51 | 15179 | 436.1 | 64.11 |
| 1D5 | 366E1 | 26.43 | 322.4 | 551.01 | 13823 | 419.27 | 101.75 |
| 1G5 | 354E2 | 19.44 | 249.47 | 308.79 | 19094 | 1378 | 86.58 |
| 1B6 | 352E1 | 13.31 | 297.87 | 473.55 | 16528 | 908.93 | 64.11 |
| 1E6 | 789E1 | 11.93 | 28.6 | 15.44 | 3144 | <70.59↓ | 273.09 |
| 1H6 | 789E2 | 9.86 | 19.33 | <12.55↓ | 6056 | <70.59↓ | 56.2 |
| 1C7 | 789E3 | 6.12 | 15.19 | <12.55↓ | 9180 | <70.59↓ | 53.33 |
| | Mean | 14.60 | 124.21 | 249.83 | 8931.95 | 643.63 | 125.95 |
| | SD | 7.72 | 127.23 | 231.22 | 6238.32 | 506.33 | 128.64 |
| 1A3 | QC1 | 252.82 | 201.08 | 210.06 | 4969 | 1984 | 1921 |
| 1C3 | QC2 | 2165 | 2013 | 2005 | 18121 | 15711 | 18072 |

### 6.5. Example 5: Proteomic Analysis of Placenta Exosomes

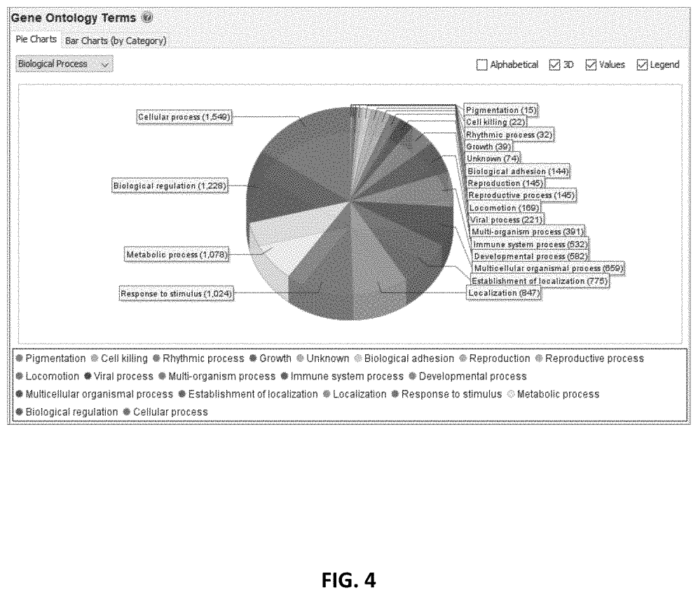

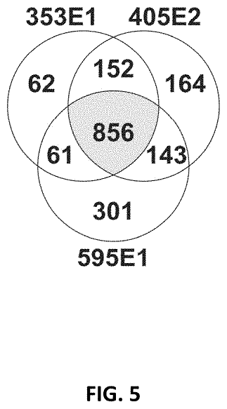

Three pExo samples were subjected to proteomic analysis. Submitted samples were lysed using a sonic probe (QSonica) with the following settings: amplitude 40%, pulse 10×1 second on, 1 second off. The protein concentration was determined by Qubit fluorometry. 10 ug of each sample was processed by SDS page and purified proteins were subject to trypsin digestion. Table 5 shows the total protein identified from each sample. Among these samples, there are total of 1814 proteins identified. Table 6 shows identification and gene ID of top identified proteins in pExo samples. Additional data is shown in FIG. 4 and FIG. 5.

TABLE 5

| | 32112 | 32113 | 32114 |
|---|---|---|---|
| Total number of proteins identified | 1313 | 1130 | 1362 |
| Total number of spectra matching | 22408 | 20850 | 23248 |
| Total number of unique peptides | 12014 | 10761 | 13380 |

TABLE 6

| | | Average |
|---|---|---|
| Identified Proteins (1814) by Proteomics in | | Relative |
| Placental Exosomes | Accession Number | Abundance |
| Cytoplasmic aconitate hydratase OS = Homo | sp\|P21399\|ACOC_HUMAN | 145 |
| sapiens GN = ACO1 PE = 1 SV = 3 | | |
| Cell surface glycoprotein MUC18 OS = Homo | sp\|P43121\|MUC18_HUMAN | 131 |
| sapiens GN = MCAM PE = 1 SV = 2 | | |
| Protein arginine N-methyltransferase 1 | sp\|Q99873\|ANM1_HUMAN | 119 |
| OS = Homo sapiens GN = PRMT1 PE = 1 SV = 2 | | |
| Guanine nucleotide-binding protein G(s) subunit | sp\|Q5JWF2\|GNAS1_HUMAN | 99 |
| alpha isoforms XLas OS = H sapiens GN = GNAS | | |
| PE = 1 SV = 2 | | |
| Cullin-5 OS = Homo sapiens GN = CUL5 PE = 1 | sp\|Q93034\|CUL5_HUMAN | 91 |
| SV = 4 | | |
| Calcium-binding protein 39 OS = Homo sapiens | sp\|Q9Y376\|CAB39_HUMAN | 83 |
| GN = CAB39 PE = 1 SV = 1 | | |
| Glucosidase 2 subunit beta OS = Homo sapiens | sp\|P14314\|GLU2B_HUMAN | 72 |
| GN = PRKCSH PE = 1 SV = 2 | | |
| Chloride intracellular channel protein 5 | sp\|Q9NZA1\|CLIC5_HUMAN | 72 |
| OS = Homo sapiens GN = CLIC5 PE = 1 SV = 3 | | |
| Semaphorin-3B OS = Homo sapiens | sp\|Q13214\|SEM3B_HUMAN | 72 |
| GN = SEMA3B PE = 2 SV = 1 | | |
| 60S ribosomal protein L22 OS = Homo sapiens | sp\|P35268\|RL22_HUMAN | 72 |
| GN = RPL22 PE = 1 SV = 2 | | |
| Spliceosome RNA helicase DDX39B OS = Homo | sp\|Q13838\|DX39B_HUMAN | 71 |
| sapiens GN = DDX39B PE = 1 SV = 1 | | |
| Transcriptional activator protein Pur-alpha | sp\|Q00577\|PURA_HUMAN | 68 |
| OS = Homo sapiens GN = PURA PE = 1 SV = 2 | | |
| Programmed cell death protein 10 OS = Homo | sp\|Q9BUL8\|PDC10_HUMAN | 66 |
| sapiens GN = PDCD10 PE = 1 SV = 1 | | |
| BRO1 domain-containing protein BROX | sp\|Q5VW32\|BROX_HUMAN | 66 |
| OS = Homo sapiens GN = BROX PE = 1 SV = 1 | | |
| Kynurenine--oxoglutarate transaminase 3 | sp\|Q6YP21\|KAT3_HUMAN | 65 |
| OS = Homo sapiens GN = KYAT3 PE = 1 SV = 1 | | |
| Laminin subunit alpha-5 OS = Homo sapiens | sp\|O15230\|LAMA5_HUMAN | 64 |
| GN = LAMA5 PE = 1 SV = 8 | | |
| ATP-binding cassette sub-family E member 1 | sp\|P61221\|ABCE1_HUMAN | 61 |
| OS = Homo sapiens GN = ABCE1 PE = 1 SV = 1 | | |
| Syntaxin-binding protein 3 OS = Homo sapiens | sp\|O00186\|STXB3_HUMAN | 60 |
| GN = STXBP3 PE = 1 SV = 2 | | |
| Proteasome subunit beta type-7 OS = Homo | sp\|Q99436\|PSB7_HUMAN | 60 |
| sapiens GN = PSMB7 PE = 1 SV = 1 | | |
| Glycogen [starch] synthase, muscle OS = Homo | sp\|P13807\|GYS1_HUMAN | 59 |
| sapiens GN = GYS1 PE = 1 SV = 2 | | |
| NAD(P)H-hydrate epimerase OS = Homo sapiens | sp\|Q8NCW5\|NNRE_HUMAN | 59 |
| GN = NAXE PE = 1 SV = 2 | | |
| Hypoxia up-regulated protein 1 OS = Homo | sp\|Q9Y4L1\|HYOU1_HUMAN | 57 |
| sapiens GN = HYOU1 PE = 1 SV = 1 | | |
| Coagulation factor XI OS = Homo sapiens | sp\|P03951\|FA11_HUMAN | 57 |
| GN = F11 PE = 1 SV = 1 | | |
| Histone H1.0 OS = Homo sapiens GN = H1F0 | sp\|P07305\|H10_HUMAN | 56 |
| PE = 1 SV = 3 | | |
| COP9 signalosome complex subunit 4 OS = Homo | sp\|Q9BT78\|CSN4_HUMAN | 56 |
| sapiens GN = COPS4 PE = 1 SV = 1 | | |
| 40S ribosomal protein S15a OS = Homo sapiens | sp\|P62244\|RS15A_HUMAN | 56 |
| GN = RPS15A PE = 1 SV = 2 | | |
| Protein ABHD11 OS = Homo sapiens | sp\|Q8NFV4\|ABHDB_HUMAN | 54 |
| GN = ABHD11 PE = 1 SV = 1 | | |
| Retinal dehydrogenase 1 OS = Homo sapiens | sp\|P00352\|AL1A1_HUMAN | 53 |
| GN = ALDH1A1 PE = 1 SV = 2 | | |
| GDP-mannose 4,6 dehydratase OS = Homo | sp\|O60547\|GMDS_HUMAN | 53 |
| sapiens GN = GMDS PE = 1 SV = 1 | | |
| Ketosamine-3-kinase OS = Homo sapiens | sp\|Q9HA64\|KT3K_HUMAN | 53 |
| GN = FN3KRP PE = 1 SV = 2 | | |
| Protein/nucleic acid deglycase DJ-1 OS = Homo | sp\|Q99497\|PARK7_HUMAN | 52 |
| sapiens GN = PARK7 PE = 1 SV = 2 | | |
| Nectin-4 OS = Homo sapiens GN = NECTIN4 | sp\|Q96NY8\|NECT4_HUMAN | 51 |
| PE = 1 SV = 1 | | |
| Cdc42-interacting protein 4 OS = Homo sapiens | sp\|Q15642\|CIP4_HUMAN | 50 |
| GN = TRIP10 PE = 1 SV = 3 | | |
| WD repeat-containing protein 61 OS = Homo | sp\|Q9GZS3\|WDR61_HUMAN | 49 |
| sapiens GN = WDR61 PE = 1 SV = 1 | | |
| CD59 glycoprotein OS = Homo sapiens | sp\|P13987\|CD59_HUMAN | 47 |
| GN = CD59 PE = 1 SV = 1 | | |
| Glycine dehydrogenase (decarboxylating), | sp\|P23378\|GCSP_HUMAN | 46 |
| mitochondrial OS = Homo sapiens GN = GLDC | | |
| PE = 1 SV = 2 | | |
| Guanine nucleotide-binding protein subunit | sp\|P29992\|GNA11_HUMAN | 43 |
| alpha-11 OS = Homo sapiens GN = GNA11 PE = 1 | | |
| SV = 2 | | |
| Serpin H1 OS = Homo sapiens GN = SERPINH1 | sp\|P50454\|SERPH_HUMAN | 42 |
| PE = 1 SV = 2 | | |
| Alpha-2-antiplasmin OS = Homo sapiens | sp\|P08697\|A2AP_HUMAN | 42 |
| GN = SERPINF2 PE = 1 SV = 3 | | |
| Heterogeneous nuclear ribonucleoprotein U | sp\|Q00839\|HNRPU_HUMAN | 42 |
| OS = Homo sapiens GN = HNRNPU PE = 1 SV = 6 | | |
| 40S ribosomal protein S11 OS = Homo sapiens | sp\|P62280\|RS11_HUMAN | 41 |
| GN = RPS11 PE = 1 SV = 3 | | |
| 3-hydroxyacyl-CoA dehydrogenase type-2 | sp\|Q99714\|HCD2_HUMAN | 41 |
| OS = Homo sapiens GN = HSD17B10 PE = 1 SV = 3 | | |
| SH3 domain-binding glutamic acid-rich-like | sp\|Q9H299\|SH3L3_HUMAN | 40 |
| protein 3 OS = Homo sapiens GN = SH3BGRL3 | | |
| PE = 1 SV = 1 | | |
| Heterogeneous nuclear ribonucleoprotein Q | sp\|O60506\|HNRPQ_HUMAN | 40 |
| OS = Homo sapiens GN = SYNCRIP PE = 1 SV = 2 | | |
| Bone marrow proteoglycan OS = Homo sapiens | sp\|P13727\|PRG2_HUMAN | 39 |
| GN = PRG2 PE = 1 SV = 2 | | |
| Lysosomal alpha-glucosidase OS = Homo sapiens | sp\|P10253\|LYAG_HUMAN | 39 |
| GN = GAA PE = 1 SV = 4 | | |
| Mannan-binding lectin serine protease 1 | sp\|P48740\|MASP1_HUMAN | 38 |
| OS = Homo sapiens GN = MASP1 PE = 1 SV = 3 | | |
| Tubulin alpha-1A chain OS = Homo sapiens | sp\|Q71U36\|TBA1A_HUMAN | 37 |
| GN = TUBA1A PE = 1 SV = 1 | | |
| CD97 antigen OS = Homo sapiens GN = CD97 | sp\|P48960\|CD97_HUMAN | 35 |
| PE = 1 SV = 4 | | |
| V-type proton ATPase subunit B, brain isoform | sp\|P21281\|VATB2_HUMAN | 35 |
| OS = Homo sapiens GN = ATP6V1B2 PE = 1 SV = 3 | | |
| von Willebrand factor A domain-containing | sp\|O00534\|VMA5A_HUMAN | 34 |
| protein 5A OS = Homo sapiens GN = VWA5A | | |
| PE = 2 SV = 2 | | |
| Integrin alpha-3 OS = Homo sapiens GN = ITGA3 | sp\|P26006\|ITA3_HUMAN | 34 |
| PE = 1 SV = 5 | | |
| Leucine--tRNA ligase, cytoplasmic OS = Homo | sp\|Q9P2J5\|SYLC_HUMAN | 34 |
| sapiens GN = LARS PE = 1 SV = 2 | | |
| Peptidyl-prolyl cis-trans isomerase FKBP3 | sp\|Q00688\|FKBP3_HUMAN | 33 |
| OS = Homo sapiens GN = FKBP3 PE = 1 SV = 1 | | |
| GTP-binding protein SAR1a OS = Homo sapiens | sp\|Q9NR31\|SAR1A_HUMAN | 33 |
| GN = SAR1A PE = 1 SV = 1 | | |
| Ras-related protein Rab-10 OS = Homo sapiens | sp\|P61026\|RAB10_HUMAN | 33 |
| GN = RAB10 PE = 1 SV = 1 | | |
| Immunoglobulin heavy variable 3-30 OS = Homo | sp\|P01768\|HV330_HUMAN | 32 |
| sapiens GN = IGHV3-30 PE = 1 SV = 2 | (+1) | |
| Ubiquitin carboxyl-terminal hydrolase 14 | sp\|P54578\|UBP14_HUMAN | 32 |
| OS = Homo sapiens GN = USP14 PE = 1 SV = 3 | | |
| Mitochondrial-processing peptidase subunit beta | sp\|O75439\|MPPB_HUMAN | 31 |
| OS = Homo sapiens GN = PMPCB PE = 1 SV = 2 | | |
| Leucyl-cystinyl aminopeptidase OS = Homo | sp\|Q9UIQ6\|LCAP_HUMAN | 31 |
| sapiens GN = LNPEP PE = 1 SV = 3 | | |
| Serine/threonine-protein kinase 10 OS = Homo | sp\|O94804\|STK10_HUMAN | 31 |
| sapiens GN = STK10 PE = 1 SV = 1 | | |
| Protein MON2 homolog OS = Homo sapiens | sp\|Q7Z3U7\|MON2_HUMAN | 31 |
| GN = MON2 PE = 1 SV = 3 | | |
| Complement component C9 OS = Homo sapiens | sp\|P02748\|CO9_HUMAN | 31 |
| GN = C9 PE = 1 SV = 2 | | |
| Heat shock protein beta-6 OS = Homo sapiens | sp\|O14558\|HSPB6_HUMAN | 31 |
| GN = HSPB6 PE = 1 SV = 2 | | |
| Complement component C8 alpha chain | sp\|P07357\|CO8A_HUMAN | 31 |
| OS = Homo sapiens GN = C8A PE = 1 SV = 2 | | |
| Tetratricopeptide repeat protein 37 OS = Homo | sp\|Q6PGP7\|TTC37_HUMAN | 30 |
| sapiens GN = TTC37 PE = 1 SV = 1 | | |
| Gasdermin-E OS = Homo sapiens GN = GSDME | sp\|O60443\|GSDME_HUMAN | 30 |
| PE = 1 SV = 2 | | |
| Acyl-protein thioesterase 1 OS = Homo sapiens | sp\|O75608\|LYPA1_HUMAN | 30 |
| GN = LYPLA1 PE = 1 SV = 1 | | |
| Exportin-1 OS = Homo sapiens GN = XPO1 PE = 1 | sp\|O14980\|XPO1_HUMAN | 29 |
| SV = 1 | | |
| Membrane cofactor protein OS = Homo sapiens | sp\|P15529\|MCP_HUMAN | 28 |
| GN = CD46 PE = 1 SV = 3 | | |
| Hydroxysteroid dehydrogenase-like protein 2 | sp\|Q6YN16\|HSDL2_HUMAN | 28 |
| OS = Homo sapiens GN = HSDL2 PE = 1 SV = 1 | | |
| ATPase ASNA1 OS = Homo sapiens GN = ASNA1 | sp\|O43681\|ASNA_HUMAN | 27 |
| PE = 1 SV = 2 | | |
| Apolipoprotein D OS = Homo sapiens GN = APOD | sp\|P05090\|APOD_HUMAN | 27 |
| PE = 1 SV = 1 | | |
| Tyrosine-protein kinase Lyn OS = Homo sapiens | sp\|P07948\|LYN_HUMAN | 27 |
| GN = LYN PE = 1 SV = 3 | | |
| Eukaryotic translation initiation factor 3 subunit | sp\|Q14152\|EIF3A_HUMAN | 27 |
| A OS = Homo sapiens GN = EIF3A PE = 1 SV = 1 | | |
| Hemopexin OS = Homo sapiens GN = HPX PE = 1 | sp\|P02790\|HEMO_HUMAN | 27 |
| SV = 2 | | |
| Target of Myb protein 1 OS = Homo sapiens | sp\|O60784\|TOM1_HUMAN | 27 |
| GN = TOM1 PE = 1 SV = 2 | | |
| EH domain-containing protein 2 OS = Homo | sp\|Q9NZN4\|EHD2_HUMAN | 26 |
| sapiens GN = EHD2 PE = 1 SV = 2 | | |
| Spectrin beta chain, erythrocytic OS = Homo | sp\|P11277\|SPTB1_HUMAN | 26 |
| sapiens GN = SPTB PE = 1 SV = 5 | | |
| L-lactate dehydrogenase B chain OS = Homo | sp\|P07195\|LDHB_HUMAN | 26 |
| sapiens GN = LDHB PE = 1 SV = 2 | | |
| Prefoldin subunit 2 OS = Homo sapiens | sp\|Q9UHV9\|PFD2_HUMAN | 26 |
| GN = PFDN2 PE = 1 SV = 1 | | |
| [Pyruvate dehydrogenase[acetyl-transferring]]- | sp\|Q9P0J1\|PDP1_HUMAN | 26 |
| phosphatase 1, mito. OS = H sapiens GN = PDP1 | | |
| PE = 1 SV = 3 | | |
| Lupus La protein OS = Homo sapiens GN = SSB | sp\|P05455\|LA_HUMAN | 26 |
| PE = 1 SV = 2 | | |
| DnaJ homolog subfamily B member 1 OS = Homo | sp\|P25685\|DNJB1_HUMAN | 26 |
| sapiens GN = DNAJB1 PE = 1 SV = 4 | | |
| Receptor expression-enhancing protein 5 | sp\|Q00765\|REEP5_HUMAN | 25 |
| OS = Homo sapiens GN = REEP5 PE = 1 SV = 3 | | |
| Calpain-1 catalytic subunit OS = Homo sapiens | sp\|P07384\|CAN1_HUMAN | 25 |
| GN = CAPN1 PE = 1 SV = 1 | | |
| 2′,3′-cyclic-nucleotide 3′-phosphodiesterase | sp\|P09543\|CN37_HUMAN | 25 |
| OS = Homo sapiens GN = CNP PE = 1 SV = 2 | | |
| Myoferlin OS = Homo sapiens GN = MYOF PE = 1 | sp\|Q9NZM1\|MYOF_HUMAN | 25 |
| SV = 1 | | |
| Plasma kallikrein OS = Homo sapiens | sp\|P03952\|KLKB1_HUMAN | 25 |
| GN = KLKB 1 PE = 1 SV = 1 | | |
| Monocyte differentiation antigen CD14 | sp\|P08571\|CD14_HUMAN | 24 |
| OS = Homo sapiens GN = CD14 PE = 1 SV = 2 | | |
| Golgin subfamily A member 3 OS = Homo sapiens | sp\|Q08378\|GOGA3_HUMAN | 24 |
| GN = GOLGA3 PE = 1 SV = 2 | | |
| Twinfilin-1 OS = Homo sapiens GN = TWF1 PE = 1 | sp\|Q12792\|TWF1_HUMAN | 24 |
| SV = 3 | | |
| Eukaryotic translation initiation factor 3 subunit | sp\|Q7L2H7\|EIF3M_HUMAN | 23 |
| M OS = Homo sapiens GN = EIF3M PE = 1 SV = 1 | | |
| Niban-like protein 1 OS = Homo sapiens | sp\|Q96TA1\|NIBL1_HUMAN | 23 |
| GN = FAM129B PE = 1 SV = 3 | | |
| Guanine nucleotide-binding protein | sp\|P62873\|GBB1_HUMAN | 23 |
| G(I)/G(S)/G(T) subunit beta-1 OS = Homo sapiens | | |
| GN = GNB1 PE = 1 SV = 3 | | |
| Galactoside-binding soluble lectin 13 OS = Homo | sp\|Q9UHV8\|PP13_HUMAN | 22 |
| sapiens GN = LGALS13 PE = 1 SV = 1 | | |
| Integrin beta-1 OS = Homo sapiens GN = ITGB1 | sp\|P05556\|ITB1_HUMAN | 22 |
| PE = 1 SV = 2 | | |
| Prostaglandin E synthase 3 OS = Homo sapiens | sp\|Q15185\|TEBP_HUMAN | 22 |
| GN = PTGES3 PE = 1 SV = 1 | | |
| Isoleucine--tRNA ligase, cytoplasmic OS = Homo | sp\|P41252\|SYIC_HUMAN | 22 |
| sapiens GN = IARS PE = 1 SV = 2 | | |
| Pregnancy-specific beta-1-glycoprotein 1 | sp\|P11464\|PSG1_HUMAN | 22 |
| OS = Homo sapiens GN = PSG1 PE = 1 SV = 1 | | |
| Adipocyte plasma membrane-associated protein | sp\|Q9HDC9\|APMAP_HUMAN | 22 |
| OS = Homo sapiens GN = APMAP PE = 1 SV = 2 | | |
| Coiled-coil domain-containing protein 93 | sp\|Q567U6\|CCD93_HUMAN | 22 |
| OS = Homo sapiens GN = CCDC93 PE = 1 SV = 2 | | |
| Protein transport protein Sec31A OS = Homo | sp\|O94979\|SC31A_HUMAN | 21 |
| sapiens GN = SEC31A PE = 1 SV = 3 | | |
| COP9 signalosome complex subunit 3 OS = Homo | sp\|Q9UNS2\|CSN3_HUMAN | 21 |
| sapiens GN = COPS3 PE = 1 SV = 3 | | |
| Uridine 5′-monophosphate synthase OS = Homo | sp\|P11172\|UMPS_HUMAN | 21 |
| sapiens GN = UMPS PE = 1 SV = 1 | | |
| Cullin-4B OS = Homo sapiens GN = CUL4B PE = 1 | sp\|Q13620\|CUL4B_HUMAN | 20 |
| SV = 4 | | |
| La-related protein 7 OS = Homo sapiens | sp\|Q4G0J3\|LARP7_HUMAN | 20 |
| GN = LARP7 PE = 1 SV = 1 | | |
| Matrix metalloproteinase-9 OS = Homo sapiens | sp\|P14780\|MMP9_HUMAN | 20 |
| GN = MMP9 PE = 1 SV = 3 | | |
| Hepatocyte growth factor activator OS = Homo | sp\|Q04756\|HGFA_HUMAN | 20 |
| sapiens GN = HGFAC PE = 1 SV = 1 | | |
| AP-2 complex subunit alpha-2 OS = Homo sapiens | sp\|O94973\|AP2A2_HUMAN | 20 |
| GN = AP2A2 PE = 1 SV = 2 | | |
| Plasma protease C1 inhibitor OS = Homo sapiens | sp\|P05155\|IC1_HUMAN | 20 |
| GN = SERPING1 PE = 1 SV = 2 | | |

### 6.6. Example 6: RNA Analysis of Placenta Exosomes

Three pExo samples were analyzed for their RNA profile by sequencing. Briefly, RNA from pExo samples are extracted and covered to cDNA and sequenced. The sequencing data is then compared to the database to identify type and identify of each sequencing data. Table 7 shows the overall profile of RNA sequencing results. The RNA in pExo contains tRNA, microRNA and other category of non-coding RNA. microRNA is the second most abundant RNA in the composition of pEXO samples. A total of 1500 different microRNA have been identified in these three pExo samples. Some commonly present in all three samples and some are uniquely present in one or two of the samples. The gene ID and relatively frequency and abundance of most abundant microRNAs are shown. MicroRNA are known to play important roles in the function of cell-cell communication.

TABLE 7

| Gene_id | Chromosome | % of Total miRNA |
|---|---|---|
| hsa-mir-26b | chr2 | 6.2606% |
| hsa-miR-26b-5p | chr2 | 6.2598% |
| hsa-mir-26a-2 | chr12 | 4.1329% |
| hsa-mir-26a-1 | chr3 | 4.1306% |
| hsa-miR-26a-5p | chr12 | 4.1306% |
| hsa-mir-30d | chr8 | 2.7200% |
| hsa-miR-30d-5p | chr8 | 2.7155% |
| hsa-mir-100 | chr11 | 2.3286% |
| hsa-miR-100-5p | chr11 | 2.3186% |
| hsa-mir-21 | chr17 | 1.5647% |
| hsa-miR-21-5p | chr17 | 1.5635% |
| hsa-mir-22 | chr17 | 1.2528% |
| hsa-miR-22-3p | chr17 | 1.2507% |
| hsa-mir-99b | chr19 | 1.2358% |
| hsa-miR-99b-5p | chr19 | 1.2230% |
| hsa-mir-181a-2 | chr9 | 1.0593% |
| hsa-mir-181a-1 | chr1 | 1.0014% |
| hsa-miR-181a-5p | chr1 | 1.0004% |
| hsa-mir-199a-2 | chr1 | 0.6194% |
| hsa-mir-199a-1 | chr19 | 0.6193% |
| hsa-mir-199b | chr9 | 0.6192% |
| hsa-miR-199a-3p | chr1 | 0.6173% |
| hsa-miR-199b-3p | chr9 | 0.6173% |
| hsa-mir-517a | chr19 | 0.8630% |
| hsa-mir-517b | chr19 | 0.8625% |
| hsa-mir-221 | chrX | 0.7610% |
| hsa-miR-221-3p | chrX | 0.7607% |
| hsa-mir-30a | chr6 | 0.7300% |
| hsa-miR-517b-3p | chr19 | 0.6874% |
| hsa-miR-517a-3p | chr19 | 0.6873% |
| hsa-mir-24-2 | chr19 | 0.7529% |
| hsa-mir-24-1 | chr9 | 0.7334% |
| hsa-miR-24-3p | chr19 | 0.7329% |
| hsa-mir-512-1 | chr19 | 0.7532% |
| hsa-mir-512-2 | chr19 | 0.7532% |
| hsa-miR-512-3p | chr19 | 0.7524% |
| hsa-mir-519a-1 | chr19 | 0.7262% |
| hsa-mir-141 | chr12 | 0.7506% |
| hsa-mir-103a-2 | chr20 | 0.6143% |
| hsa-miR-103a-3p | chr20 | 0.6130% |
| hsa-mir-103a-1 | chr5 | 0.6130% |
| hsa-miR-141-3p | chr12 | 0.7479% |
| hsa-miR-30a-5p | chr6 | 0.6009% |
| hsa-mir-200c | chr12 | 0.6287% |
| hsa-miR-200c-3p | chr12 | 0.6286% |
| hsa-mir-148a | chr7 | 0.3417% |
| hsa-miR-148a-3p | chr7 | 0.3408% |
| hsa-mir-519c | chr19 | 0.6193% |
| hsa-mir-516b-1 | chr19 | 0.7180% |
| hsa-miR-516b-5p | chr19 | 0.7178% |
| hsa-mir-518e | chr19 | 0.5433% |
| hsa-miR-320a | chr8 | 0.9335% |
| hsa-mir-320a | chr8 | 0.9335% |
| hsa-mir-522 | chr19 | 0.5108% |
| hsa-mir-23a | chr19 | 0.3359% |
| hsa-miR-23a-3p | chr19 | 0.3356% |
| hsa-mir-27b | chr9 | 0.3544% |
| hsa-miR-27b-3p | chr9 | 0.3525% |
| hsa-mir-519b | chr19 | 0.4531% |
| hsa-mir-523 | chr19 | 0.4546% |
| hsa-miR-519a-5p | chr19 | 0.4557% |
| hsa-mir-517c | chr19 | 0.3725% |
| hsa-mir-486 | chr8 | 0.4035% |
| hsa-miR-486-5p | chr8 | 0.4028% |
| hsa-miR-519b-5p | chr19 | 0.4490% |
| hsa-miR-519c-5p | chr19 | 0.4490% |
| hsa-miR-522-5p | chr19 | 0.4490% |
| hsa-miR-523-5p | chr19 | 0.4490% |
| hsa-miR-518e-5p | chr19 | 0.4487% |
| hsa-mir-143 | chr5 | 0.2889% |
| hsa-miR-143-3p | chr5 | 0.2887% |
| hsa-mir-516b-2 | chr19 | 0.5721% |
| hsa-mir-519a-2 | chr19 | 0.2933% |
| hsa-mir-10b | chr2 | 0.2067% |
| hsa-miR-10b-5p | chr2 | 0.2065% |
| hsa-miR-519a-3p | chr19 | 0.2704% |
| hsa-mir-30e | chr1 | 0.2635% |
| hsa-mir-92a-1 | chr13 | 0.3218% |
| hsa-mir-516a-1 | chr19 | 0.2681% |
| hsa-mir-516a-2 | chr19 | 0.2681% |
| hsa-miR-516a-5p | chr19 | 0.2676% |
| hsa-let-7a-3 | chr22 | 0.3538% |
| hsa-let-7a-1 | chr9 | 0.3546% |
| hsa-let-7a-5p | chr11 | 0.3544% |
| hsa-let-7a-2 | chr11 | 0.3529% |
| hsa-mir-424 | chrX | 0.2370% |
| hsa-miR-92a-3p | chr13 | 0.2961% |
| hsa-mir-92a-2 | chrX | 0.2961% |
| hsa-mir-93 | chr7 | 0.2251% |
| hsa-miR-93-5p | chr7 | 0.2249% |
| hsa-mir-526b | chr19 | 0.2720% |
| hsa-miR-1323 | chr19 | 0.3653% |
| hsa-mir-1323 | chr19 | 0.3653% |
| hsa-miR-526b-5p | chr19 | 0.2701% |
| hsa-let-7f-2 | chrX | 0.2072% |
| hsa-let-7f-5p | chr9 | 0.2072% |
| hsa-let-7f-1 | chr9 | 0.2055% |
| hsa-miR-517c-3p | chr19 | 0.1967% |
| hsa-let-7b | chr22 | 0.2197% |
| hsa-let-7b-5p | chr22 | 0.2197% |
| hsa-mir-151a | chr8 | 0.2002% |
| hsa-miR-519c-3p | chr19 | 0.1702% |
| hsa-mir-148b | chr12 | 0.1442% |
| hsa-miR-107 | chr10 | 0.1520% |
| hsa-mir-107 | chr10 | 0.1520% |
| hsa-miR-148b-3p | chr12 | 0.1411% |
| hsa-let-7i | chr12 | 0.1502% |
| hsa-let-7i-5p | chr12 | 0.1502% |
| hsa-miR-101-3p | chr1 | 0.1174% |
| hsa-mir-101-2 | chr9 | 0.1174% |
| hsa-mir-101-1 | chr1 | 0.1162% |
| hsa-miR-424-3p | chrX | 0.1552% |
| hsa-mir-519d | chr19 | 0.1433% |
| hsa-mir-27a | chr19 | 0.1629% |
| hsa-miR-517-5p | chr19 | 0.1751% |
| hsa-miR-27a-3p | chr19 | 0.1583% |
| hsa-mir-23b | chr9 | 0.1206% |
| hsa-miR-23b-3p | chr9 | 0.1205% |
| hsa-mir-10a | chr17 | 0.0945% |
| hsa-miR-10a-5p | chr17 | 0.0936% |
| hsa-miR-30e-3p | chr1 | 0.1370% |
| hsa-mir-1283-2 | chr19 | 0.1558% |
| hsa-miR-30e-5p | chr1 | 0.1264% |
| hsa-miR-30a-3p | chr6 | 0.1291% |
| hsa-mir-191 | chr3 | 0.1309% |
| hsa-miR-191-5p | chr3 | 0.1305% |
| hsa-miR-1283 | chr19 | 0.1416% |
| hsa-mir-1283-1 | chr19 | 0.1416% |
| hsa-mir-423 | chr17 | 0.1596% |
| hsa-mir-520a | chr19 | 0.1325% |
| hsa-miR-151a-3p | chr8 | 0.1290% |
| hsa-mir-520d | chr19 | 0.1287% |
| hsa-miR-520d-3p | chr19 | 0.1263% |
| hsa-miR-520a-3p | chr19 | 0.1242% |
| hsa-mir-518c | chr19 | 0.1092% |
| hsa-miR-519d | chr19 | 0.1026% |
| hsa-mir-335 | chr7 | 0.0681% |
| hsa-mir-524 | chr19 | 0.1320% |
| hsa-mir-16-2 | chr3 | 0.0867% |
| hsa-mir-25 | chr7 | 0.1007% |
| hsa-miR-25-3p | chr7 | 0.1005% |
| hsa-miR-335-5p | chr7 | 0.0645% |
| hsa-mir-16-1 | chr13 | 0.0833% |
| hsa-miR-16-5p | chr13 | 0.0829% |
| hsa-miR-192-5p | chr11 | 0.0956% |
| hsa-mir-192 | chr11 | 0.0956% |
| hsa-miR-518c-3p | chr19 | 0.0930% |
| hsa-miR-423-3p | chr17 | 0.1019% |
| hsa-miR-424-5p | chrX | 0.0818% |
| hsa-mir-140 | chr16 | 0.0914% |
| hsa-miR-320b | chr1 | 0.1382% |
| hsa-mir-320b-2 | chr1 | 0.1382% |
| hsa-mir-320b-1 | chr1 | 0.1374% |
| hsa-miR-140-3p | chr16 | 0.0873% |
| hsa-miR-518e-3p | chr19 | 0.0946% |
| hsa-mir-518b | chr19 | 0.0883% |
| hsa-let-7g | chr3 | 0.0762% |
| hsa-let-7g-5p | chr3 | 0.0762% |
| hsa-miR-518b | chr19 | 0.0823% |
| hsa-miR-222-3p | chrX | 0.0874% |
| hsa-mir-222 | chrX | 0.0875% |
| hsa-miR-524-3p | chr19 | 0.1032% |
| hsa-miR-20a-5p | chr13 | 0.0595% |
| hsa-mir-20a | chr13 | 0.0595% |
| hsa-miR-151a-5p | chr8 | 0.0712% |
| hsa-miR-186-5p | chr1 | 0.0752% |
| hsa-mir-186 | chr1 | 0.0752% |
| hsa-mir-660 | chrX | 0.0606% |
| hsa-miR-660-5p | chrX | 0.0604% |
| hsa-mir-125a | chr19 | 0.0953% |
| hsa-miR-203a | chr14 | 0.0536% |
| hsa-mir-203a | chr14 | 0.0536% |
| hsa-mir-106b | chr7 | 0.0669% |
| hsa-mir-520g | chr19 | 0.0731% |
| hsa-miR-451a | chr17 | 0.0587% |
| hsa-mir-451a | chr17 | 0.0589% |
| hsa-miR-522-3p | chr19 | 0.0618% |
| hsa-mir-378a | chr5 | 0.0840% |
| hsa-mir-30b | chr8 | 0.0724% |
| hsa-miR-181a-2-3p | chr9 | 0.0589% |
| hsa-mir-181b-2 | chr9 | 0.0656% |
| hsa-miR-378a-3p | chr5 | 0.0836% |
| hsa-miR-181b-5p | chr1 | 0.0650% |
| hsa-miR-125a-5p | chr19 | 0.0842% |
| hsa-mir-584 | chr5 | 0.0728% |
| hsa-miR-584-5p | chr5 | 0.0728% |
| hsa-miR-29a-3p | chr7 | 0.0496% |
| hsa-mir-29a | chr7 | 0.0497% |
| hsa-mir-518a-1 | chr19 | 0.0680% |
| hsa-mir-518a-2 | chr19 | 0.0680% |
| hsa-mir-181b-1 | chr1 | 0.0616% |
| hsa-miR-30b-5p | chr8 | 0.0685% |
| hsa-miR-518a-3p | chr19 | 0.0662% |
| hsa-mir-28 | chr3 | 0.0567% |
| hsa-mir-146b | chr10 | 0.0609% |
| hsa-miR-146b-5p | chr10 | 0.0607% |
| hsa-miR-520g | chr19 | 0.0636% |
| hsa-mir-515-1 | chr19 | 0.0543% |
| hsa-mir-515-2 | chr19 | 0.0543% |
| hsa-miR-106b-3p | chr7 | 0.0554% |
| hsa-mir-30c-2 | chr6 | 0.0559% |
| hsa-mir-30c-1 | chr1 | 0.0555% |
| hsa-miR-30c-5p | chr1 | 0.0547% |
| hsa-mir-518f | chr19 | 0.0510% |

### 6.7. Example 7: Placenta Exosome Promotes Migration of Human Dermal Fibroblast Cells (HDF)

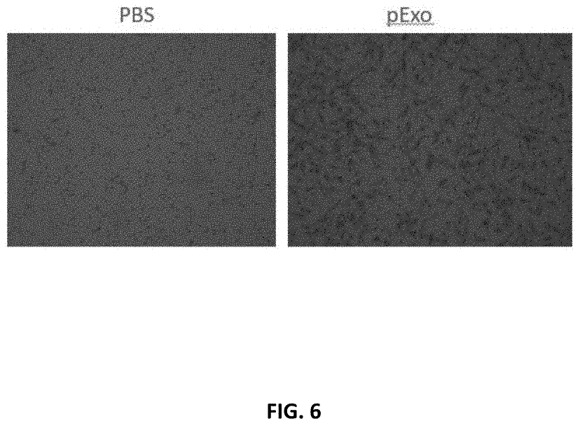

The cytokine profile shows pExo include chemotactic growth factors, suggesting that pExo should have the function to promote cell migration. To examine this, transwell migration assay was set up as the following: 750 uL of DMEM basal medium (without serum) was placed on the bottom chamber of a transwell (24-well) plate, pExo was added at 50 uL. PBS was added at the same volume as control. 1×10e5 HDF were seeded on the top chamber of the transwells (8 um pore). After 6 to 24 hours, the cells on the top chamber of the transwell were removed by cotton swab. The transwells are then fixed in solution containing 1% ethanol in PBS, followed by stained with 1% crystal violet dissolved in 1% ethanol-PBS. The migrated cells are visualized with microscope. The data shows the example of results of HDF migrated to the bottom side of the transwell while there was significantly less cell migrated through the well in the PBS control transwell. The study demonstrates that pExo can promote the migration of human dermal fibroblast cells. See, FIG. 6.

### 6.8. Example 8: Placenta Exosomes Promote Migration of Human Umbilical Cord Blood Endothelial Cells (HUVECs)

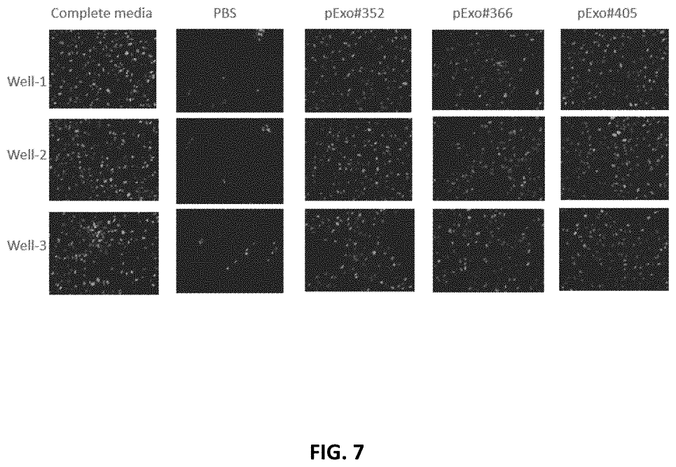

Transwell migration assay was also set up as the following: 750 uL of DMEM basal medium (without serum) was placed on the bottom chamber of a transwell (24-well) plate, pExo was added at 50 uL. PBS was added at the same volume as control. 2×10e5 HUVEC expressing GFP proteins were seeded on the top chamber of the transwells (8 um pore). After 6 to 24 hours, the migrated wells are visualized directly with an inverted fluorescence microscope (AMG). The study demonstrates that all three pExo sample tested can promote the migration of HUVEC in all three duplicated wells. Complete medium for HUVEC is used as a positive control has significant cell migration and PBS is used as an additional control has significantly less cell migrated through comparing with complete media or pExo tested wells. See, FIG. 7.

### 6.9. Example 9: Placenta Exosomes Stimulate Proliferation of HUVECs

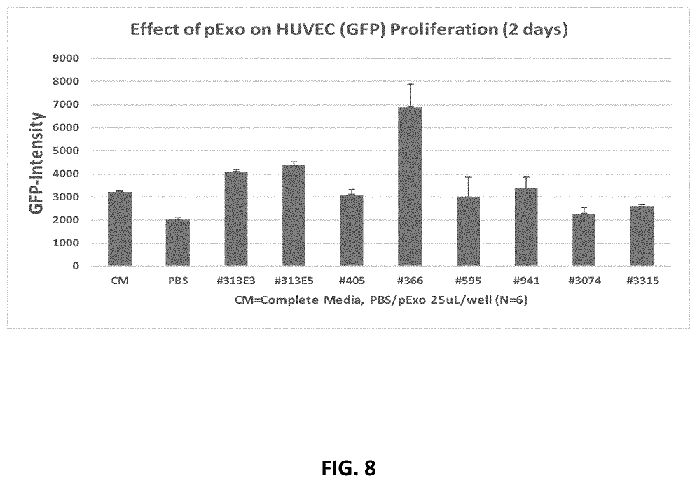

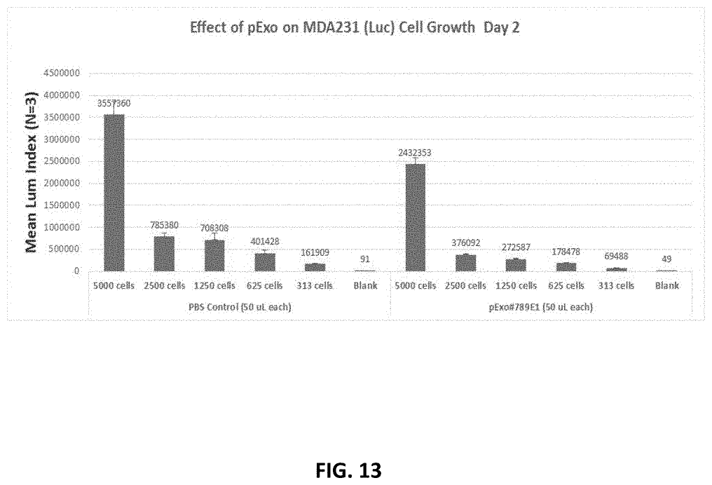

Cytokine profiles of pExo shows it has several growth factors (PGDF-AA,BB, VEGF) that are known to be involved in the growth of HUVECs. To examine the effect of pExo on the growth and proliferation of HUVEC. HUVEC expressing GFP were seeded at 1×10e4 cells in 96-well plate (transparent bottom and non-transparent walls) in 100 uL of complete HUVEC growth medium. After seeding for 2 hours, cells were attached to the bottom of the wells. The wells are then added with 25 uL of different pExo samples (N=6 per sample). The plate is then evaluated with their fluorescence intensity using a plate reader (Synergy H4, excitation 395 nm/emission 509 nm) at day-0 and day-2 after seeding. As shown in FIG. 13, Complete media demonstrate higher GFP signals (indicator of cell number) from day-0 to day-2. PBS control, in which the complete medium is 50% diluted, showed slight growth comparing with complete media. All eight different pExo samples all showed higher growth of GFP at day 2. See, FIG. 8.

### 6.10. Example 10: Placenta Exosomes Stimulate Proliferation and Colony Formatioin of Human CD34+ Cells

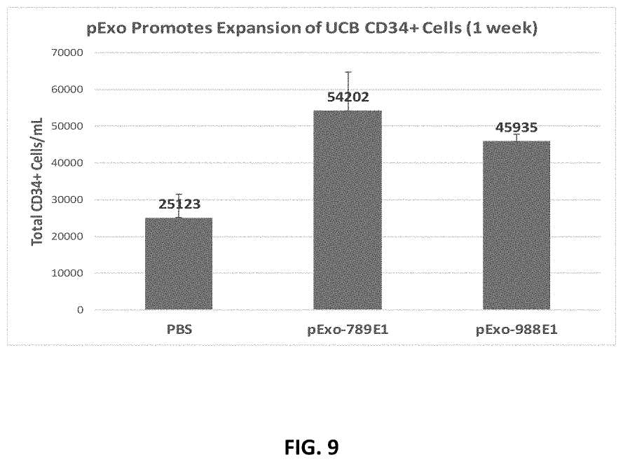

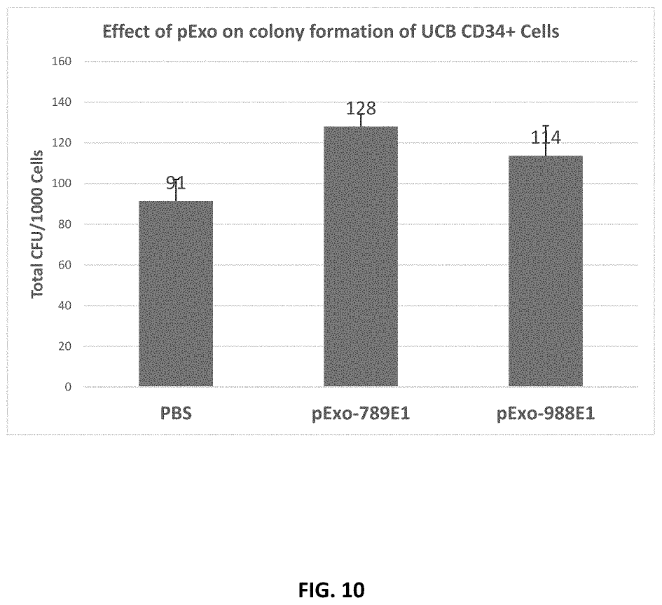

To test the effects of pExo on the proliferation of hematopoietic stem cells, human umbilical cord blood CD34+ cells (prepared in house) were thawed and cultured in expansion medium containing a cocktail of SCF, Flt-3, KL (medium A) with 10% FCS-IMDM at 1×10e4/cells per ml (N=4). Culture wells were added with either 25 uL of PBS or 25 uL of pExo samples (two pExo samples tested). After one week of culture, the total cell number of each well was counted and the percentage of CD34+ cells in the culture was evaluated by flow cytometry (FACS) using anti-CD34 antibodies. The total CD34+ cell number is calculated as the total cell number in the well to the % of CD34+ cell in the culture. The results showed both pExo treated culture has significantly higher number of CD34+ cells comparing with PBS control culture. pExo was also tested on their effect on CD34+ cells in a colony forming unit culture (CFU). CFU cultures were established with MethoCult H4434 media (Stem Cell Technologies) and pExo or PBS was added at 50 uL/mL. After two weeks of culture, the total CFU number in each 35-mm dish is counted (N=3). The data showed that at the presence of pExo, there are significantly higher number of CFU comparing with PBS control cultures. See, FIG. 9 and FIG. 10.

### 6.11. Example 11: Inhibition of Cancer Cell Proliferation

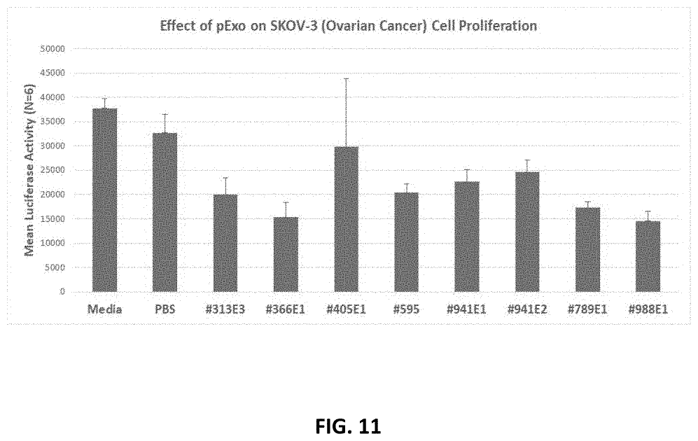

MicroRNA data and cytokine data suggest that pExo have the activities to inhibit cancer cell proliferation. pExo samples was used to examine its effect on the growth of SKOV3 (Human ovarian cancer cell line) in 96-well plate. This SKOV3 cells is engineered to express Luciferase, therefore, measuring the luciferase activity is an index of cell growth. A total of 8 different pExo samples were used. 2000 SKOV3 cells were added to 96-well plate in 100 uL of growth medium (DMEM-10% FCS). 2 hrs later, 40 uL of pExo was added to the well (N=6) and supplemented with 60 uL, of growth media. 40 uL of PBS was used as control. The complete medium condition is by adding 100 uL of medium to the wells. After culturing for 2 days in incubator, the activity of the Luciferase are measured with Luciferase Assay Kit (Promega) by lysed the cells and the Luciferase activity was measured with the Luminescence emission with a plate reader (Synergy H4). The data shows that at each cell concentration, pExo treated culture had significantly less Luminex index comparing with PBS control. This data indicates that pExo inhibited the growth of SKOV3 cells. See, FIG. 11.

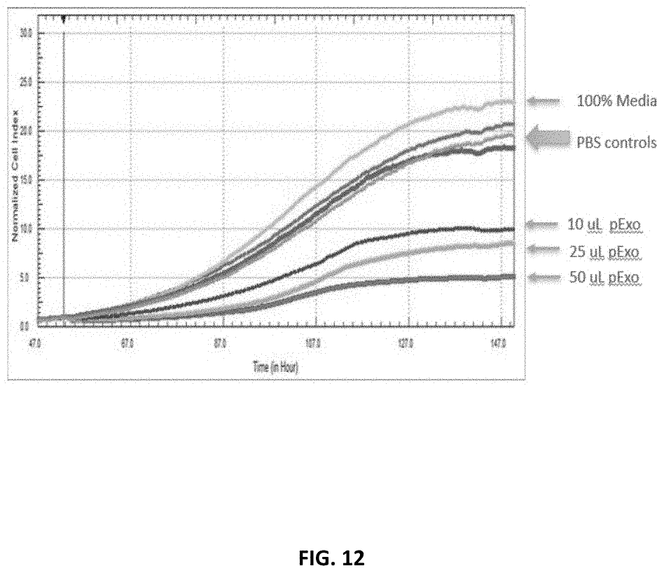

A549 cancer cell line (a human lung carcinoma cancer cell line) was seeded at 1500 cells/well in a 96-well plate (Xiceligence). After seeding 24 hrs, pExo are added at three difference dose (5 uL, 25 and 50 uL) in the growth media (100 uL). Same amount of PBS was added as control. The growth of the cells can be monitored from day 1 to day 3 after seeding through the software that reflect the adherence of the cells on wells. The data showed that at the presence of pExo, the growth of the cells, as shown as normalized cell index, was significantly lower at the presence of pExo comparing with PBS controls. Each of the growth curve is the average cell index from three independent wells. See FIG. 12.

pExo sample was used to examine its effect on the growth of MDA231 (Human breast cancer cell line) in 96-well plate with different cell doses. This MDA231 cells is engineered to express Luciferase, therefore, measuring the luciferase activity is an index of cell growth. Different cell number of MDA231-Luciferase is seeded to 96-well plates (triplicates) and added with 25 uL of pExo #789. After culturing for 2 days in incubator, the activity of Luciferase is measured with Luciferase Assay Kit (Promega) by lysed the cells and the Luciferase activity was measured with the Luminescence emission with a plate reader (Synergy H4). The data shows that at each cell concentration, pExo treated culture had significantly less Luminex index comparing with PBS control. This data indicates that pExo inhibited the growth of MDA231 cells. See, FIG. 13.

### 6.12. Example 12: Placenta Exosomes Modulate Activation and Differentiation of Immune Cells

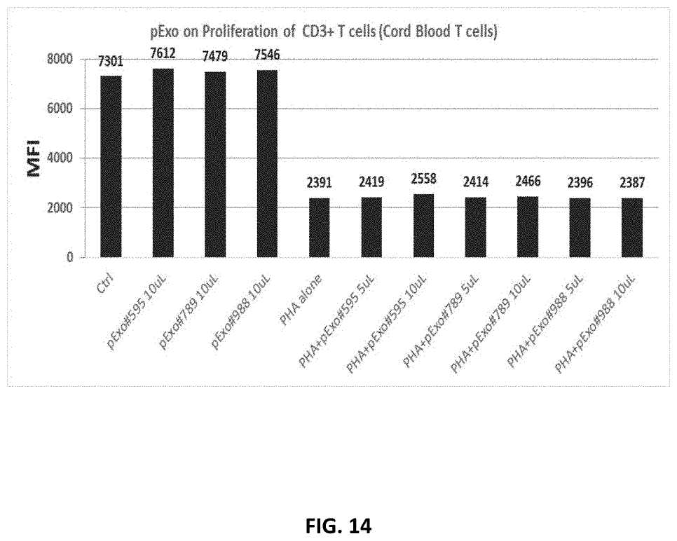

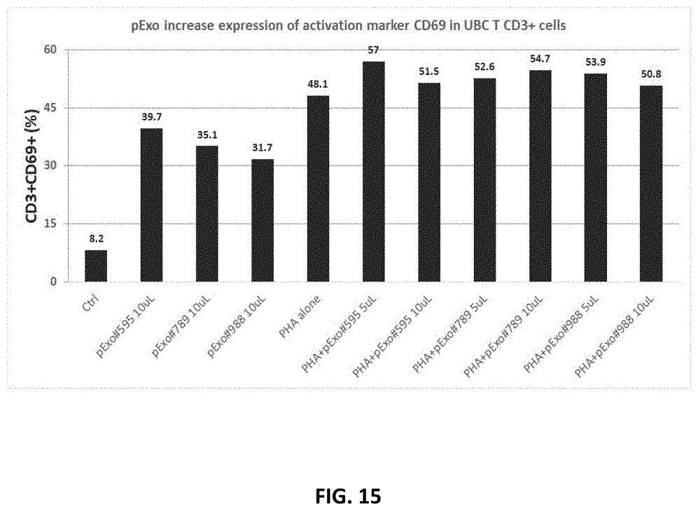

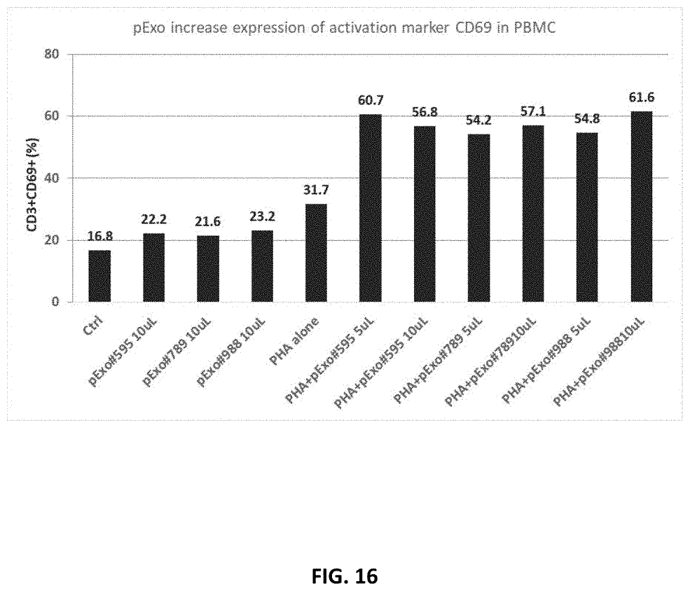

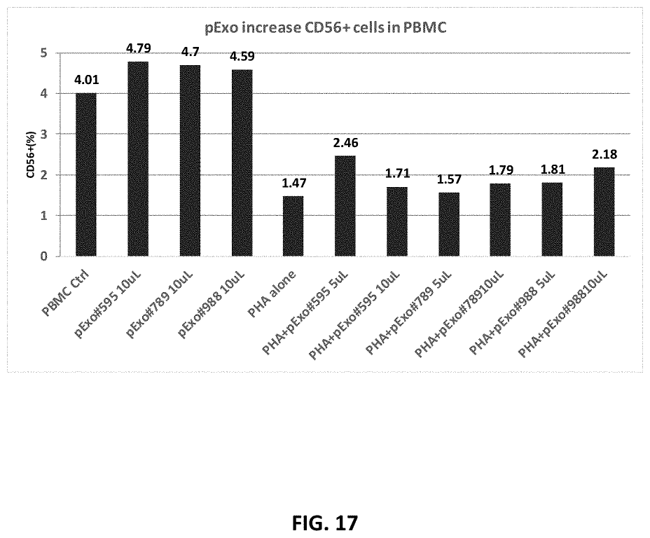

To examine the effect of pExo on immune cells, human umbilical cord blood T cells were labeled with PKH Fluorescence dye and then incubated with pExo or PHA as stimulation. After culturing in RPMI+10% FCS for 5 days, cells are analyzed with FACS with antibodies that can distinguish total T cells as well as subtypes of different type of T cells including CD4, CD8, CD69, CD27. The data shows that at the presence of pExo, the MFI of CD3+ cells are similar to control culture, indicating that pExo alone do not affect the proliferation activity on the T cells. At PHA stimulation, the MFI significantly reduced, indicating that the cells proliferated, at the presence of both PHA and pExo, WI is similar to PHA alone, indicating that the cell proliferation is not affected by the presence of pExo. It was found that CD69+ cells are significantly higher in cells treated with pExo, CD69+ cells significantly increased in CD3+ cells (T cells), indicating that T cell activation was increased by pExo. This observation was found in both cord blood T cells and PBMC cells. In addition, pExo was found to increase the percentage of CD56+ cells (NK) cells in PBMC. See, FIG. 14, FIG. 15, FIG. 16, and FIG. 17.

### 6.13. Example 13: Yield of Exosomes from Cultivated Placenta, Placenta Perfusate and PRP (Cord Blood Serum)

Placenta perfusate and PRP (cord blood serum) were isolated by the same method of cultivated human placenta tissues. The table below shows the yield of exosome from the placenta perfusate and PRP are significantly less than cultivated placenta.

TABLE 8
Yield of exosomes (mg) isolated from Placenta perfusate,
PRP and Cultivated Placenta

| Samples/Source | Perfusate | PRP | Cultivated Placenta |
|---|---|---|---|
| 1 | 0.30 | 0.07 | 114.7 |
| 2 | 0.02 | 0.39 | 88.8 |
| 3 | 0.21 | 0.67 | 103.4 |
| 4 | 0.25 | 0.47 | 70.0 |
| 5 | 0.36 | | 63.1 |
| 6 | 1.35 | | 97.45 |
| 7 | 0.23 | | 70.46 |
| Mean | 0.39 | 0.40 | 86.84 |
| SD | 0.44 | 0.25 | 19.50 |

### DISCUSSION

The subject methods are capable of producing large amounts of exosomes with unique and advantageous properties. The exosomes are shown to contain many proteins and RNAs which, due to the demonstrated function of the exosomes are believed to be bioactive. The exosomes express many cell surface markers with may act as binding partners, e.g., as a receptor or ligand, and thereby allow targeting of this biological activity to desired cell types.

The data presented herein show utility for the exosomes of the for a wide variety of indications such as those described in Table 9.

TABLE 9

| Functional | | |
|---|---|---|
| Regeneration | | |
| Indication | | |
| Targets of pExo | Rationales | References |
| Functional | pExo contains cytokines and | |
| regeneration | growth factors that are | |
| including but not | involved in chemotaxis. | |
| limiting to: | pExo showed activity of | |
| stroke, Spinal | enhance cell migration. | |
| cord injury, skin | pExo showed activity in the | |
| lesions, wound | stimulation of HUVEC cell | |
| healing, acute | proliferation. | |
| and chronic | | |
| myocardial | | |
| infarction | | |
| Orthopedic, | pExo contains cytokines and | |
| cosmetic and | growth factors that are | |
| regenerative | involved in chemotaxis. | |
| medicine | pExo showed activity of | |
| applications | enhance cell migration. | |
| | pExo showed activity in the | |
| | stimulation of HUVEC cell | |
| | proliferation. | |
| Anti-aging | pExo contains cytokines and | |
| applications | growth factors that are | |
| | involved in chemotaxis. | |
| | pExo showed activity of | |
| | enhance cell migration. | |
| | pExo showed activity in the | |
| | stimulation of HUVEC cell | |
| | proliferation. | |
| Hair | pExo contains cytokines and | |
| regeneration | growth factors that are | |
| | involved in chemotaxis. | |
| | pExo showed activity of | |
| | enhance cell migration. | |
| | pExo showed activity in the | |
| | stimulation of HUVEC cell | |
| | proliferation. | |
| Organ failure | pExo contains cytokines and | |
| | growth factors that are | |
| | involved in chemotaxis. | |
| | pExo showed activity of | |
| | enhance cell migration. | |
| | pExo showed activity in the | |
| | stimulation of HUVEC cell | |
| | proliferation. | |
| Vascular | pExo contains cytokines and | |
| disorders | growth factors that are | |
| | involved in chemotaxis. | |
| | pExo showed activity of | |
| | enhance cell migration. | |
| | pExo showed activity in the | |
| | stimulation of HUVEC cell | |
| | proliferation. | |
| Erectile | pExo contains VEGF, | Xie et al. (2008). Growth factors for |
| dysfunction | PDGF, FGF2 which are pro- | therapeutic angiogenesis in |
| | angiogenesis. Degeneration | hypercholesterolemic erectile dysfunction. |
| | in the vasculature bed can | Asian J Androl. 10: 23-7 |
| | result in erectile dysfuntion. | |
| | pExo can enhance | |
| | angiogenesis. | |
| Protection for | pExo contains FGF2. FGF2 | Kinoda J. et al. (2018). Protective effect of |
| radiation | were demonstrated to have | FGF2 and low molecular-weight |
| induced wound | protective effect on | heparin/protamine nanoparticles on |
| repair | radiation-induced healing- | ratiation-induced healing-impaired wound |
| | impaired wound repair in | repair in rats. J. Radiat Res. 59: 27-34. |
| | rats. | |
| Axonal | pExo contains FGF2. FGF2 | Nagashima et al. (2017). Sci Rep. Priming |
| regeneration and | were demonstrated to have | with FGF2 stimulates human dental pulp |
| locomotor | the activity to stimulate | cells to promote axonal regeneration and |
| function | human dental pulp cells to | locomotor function recovery after spinal |
| recovery after | promote axonal | cord injury. 7: 13500. |
| Spinal cord | regeneration and locomotor | |
| injury | fuction recovery after spinal | |
| | cord injury. | |
| Liver diseases | pExo contains FGF2. FGF2 | Sato-Matsubara et al. (2017) et al. |
| | were demonstrated to have | Fibroblast growth factor-2 regulates |
| | the activity to stimulate | cytoglobin expression and activation of |
| | cytoglobin expression and | human hepatic stellate cells via JNK |
| | activation of human hepatic | signaling. J. Biol Chem. 292: 18961-18972. |
| | stellate cells. | |
| Axonal | pExo contains FGF2. FGF2 | Lee et al. (2017). Recombinant human |
| regeneration and | were demonstrated to have | fibroblast growth factor-2 promotes nerve |
| locomotor | the activity to promote | regeneration and functional recovery after |
| function | nerve regeneration and | mental nerve crush injury. Neural Regen |
| recovery after | fuctional recovery after | Res. 12: 629-636. |
| Spinal cord | mental nerve crush injury. | |
| injury | | |
| Polycystic overy | pExo contains Fractalkine. | Huang et al. (2016). Fractalkine restore the |
| syndrome | Fractalkine were | decreased expression of StAR and |
| | demonstrated to have the | progesterone in granulosa cells from |
| | activity to restore the | patients with polycystic ovary syndrome. |
| | expression of StAR and | Sci. Rep. 6: 26205. |
| | progesterone in granulosa | |
| | cells from patients with | |
| | polycystic ovary syndrome. | |
| Periodontal | pExo contains FGF2 and | Li et al. (2018). Evaluation of recombinant |
| regeneration | PDGF-BB. FGF2 and | human FGF-2 and PDGF-BB in periodontal |
| | PDGF-BB can enhance | regeneration: A systemic review and meta- |
| | peridontal diseases. | analysis. Sci Rep. 7: 65.. |
| Hair growth | pExo contains FGF2 and | Bak et al. (2018) Human umbilical cord |
| | PDGF-BB, VEGF. | blood mesenchymal stem cells engineered |
| | | to overexpress growth factors accelerate |
| | | outcomes in hair growth. Korea J. Physiol |
| | | Pharmcol. 22: 555-566. |
| Axonal | pExo contains micro-RNA | Sun et al. (2018). Network analysis of |
| regeneration and | MIR-26a-5p, which have | microRNAs, transcription factors, and target |
| locomotor | been implicated in the axon | genes involved in axon regeneration. J |
| function | regeneration. | Zhejiang Univer. Sci. 19: 293-304. |
| recovery after | | |
| Spinal cord | | |
| injury | | |
| Anti Cancer | | |
| Indication | pExo contains anti-tumor | |
| Targets of pExo | micro-RNA below | |
| Anti-tumor | microRNA-26b: microRNA | Li YP et al. (2017). Effects of microRNA- |
| treatments | (miR)-26b inhibits | 26b on proliferation and invatioin of glioma |
| including all | neuroglioma (U87 glioma | cells and related mechanisms. Mol Med Rep |
| different types of | cells) | 16: 4165-4170. |
| cancers eg. | | |
| Neuroglioma | | |
| Anti-tumor | microRNA-26b: represses | Zhang Y et al (2014). MicroRNA-26b |
| treatments | colon cancer cell | represses colon cancer cell proliferation by |
| including all | proliferation | inhibiting lymphoid enhancer factor 1 |
| different types of | | expression. Mol Cancer Ther. 13: 1942-51. |
| cancers eg. | | |
| Colan cancer | | |
| Anti-tumor | microRNA-26b-5p: | Fan et al. (2018). MicroRNA-26-5p |
| treatments | inhibiting human | regulates cell proliferation, invasion, and |
| including all | intrahepatic | metastasis in human intrahepatic |
| different types of | cholangiocarcinoma tumor | cholangiocarcinoma by targeting S100A7. |
| cancers: eg. | cell lines RBE and HCCC- | Oncol Lett. 15: 386-392. |
| Liver cancer | 9810. | |
| Anti-tumor | microRNA-26-a-5p and | Niyamoto et al (2016). Tumor-suppressive |
| treatments | microRNA-26b-5p inhibits | miRNA-26a-5p and miR-26-5p inhibit cell |
| including all | growth of bladder cancer | aggressiveness by regulating PLOD2 in |
| different types of | cells. | bladder cancer. |
| cancers. Eg. | | |
| Blader cancer | | |
| Anti-tumor | microRAN-26b-5p inhibits | Wang Y et al. (2016). Regulation of |
| treatments | hepatocellular carcinoma | proliferation, angiogenesis and apoptosis in |
| including all | | hepatocellular carcinoma by miR-26b-5p. |
| different types of | | Tumor Biol. 37: 10965-79. |
| cancers | | |
| Anti-tumor | mir-22 supppress | Zhang X et al. (2017). miR-22 suppress |
| treatments | tumorgenesis in breast | tumorigenesis and improves radiosensitivity |
| including all | cancer | of breast cancer cells by targeting Sirt1. |
| different types of | | Biol Res. 50: 27. |
| cancers | | |
| Anti-tumor | mic-22 suppress colon | Xia SS et al. (2017). MciroRNA-22 |
| treatments | cancer cells | suppresses the growth, migration, and |
| including all | | invasion of colorectal cancer celsl through a |
| different types of | | Sp1 negative feedback loop. Oncotarget. |
| cancers | | 30: 36266-36278. |
| Anti-tumor | MiR-99B and Mir-99-B-5P | Li W et al. (2015). miRNA-99-5p |
| treatments | inhibits metastasis of | suppresses liver metastasis of colorectal |
| including all | colorectal cancer cells to | cancer by down-regulating mTOR. |
| different types of | liver | Oncotarget 6: 24448-62. |
| cancers | | |
| Anti-tumor | mir-181a and mir-181b | Shi et al. (2008). Has-mir-181a and has-mir- |
| treatments | suppress human glioma | 181b functions as tumor suppressors in |
| including all | cells trigers growth | human glioma cells. Brain Res. 1236: 185-93. |
| different types of | inhibition, induced | |
| cancers | apoptosis and inhibited | |
| | invation in glioma cels. | |
| Anti-tumor | Mir-199a-2, mir-199-a1, | Koshizuka et al. (2017). Regulation of |
| treatments | mir-199-B, mir-199A-1p, | ITGA3 by the anti-tumor miR-199 family |
| including all | mir-199b-3p micro RNAs | inhibits cancer migration and invation in |
| different types of | are anti-tumor miR199 | head and neck cancer. Cancer Sci. |
| cancers | family that inhibits cancer | 108: 1681-1692. |
| | cell migration and invation | |
| | in head and neck cancer | |
| Anti-tumor | Mir-221 and Mir-221-2p | Xie et al. (2018) MIR-221 inhibits |
| treatments | inhibits proliferation of | proliferation of pancreatic cell cells via |
| including all | pancreatic cancer cells | down regualtion of SOCS3. Eur Rev Med |
| different types of | | Pharmacol Sci. 22: 1914-1921. |
| cancers | | |
| Anti-tumor | MircoRNA-30a inhibits | Liu YC et al. (2017) MicroRNA-30a |
| treatments | colorectal cancer metastasis | inhibits colorectal cancer metastasis through |
| including all | through down-regulation of | down regulation of type 1 insulin like |
| different types of | type 1 insulin-like growth | growth factor receptor. |
| cancers | factor receptor | |
| Anti-tumor | miR-130-a-3p inhibits | Kong et al. (2018). MiR-130-3p inhibits |
| treatments | migration and invation in | migration and invation by regulating |
| including all | human breast cancer stem | RAB5B in human breast cancer stem cell- |
| different types of | cell-like cells | like cells. Biochem Biophys Res Commun. |
| cancers | | 501: 486-493. |
| Anti-tumor | miR-24-2 inhibits breast | Manvati et al. (2105). miR-24-2 regulates |
| treatments | cancer cells growth. | genes in survival pathway and demonstrates |
| including all | | potentials in reducing cellular viability in |
| different types of | | combination with docetaxel. Gene. 10: 217-24. |
| cancers: eg. | | |
| Breast cancer | | |
| Anti-tumor | miR-24-2 inhibits growth of | Pandita et al. (2015). Combined effect of |
| treatments | pancreatic cancer cell lines | microRNA, nutraceuticals and drug on |
| including all | | pancreatic cancer cell lines. Chem Biol |
| different types of | | Interact. 233: 56-64. |
| cancers: eg. | | |
| Pancreatic | | |
| cancer | | |
| Anti-tumor | microRNA-24-1 inhibits | Liu Y et al. (2017). MicroRNA-24-1 |
| treatments | hepatomal cell invasion and | suppress mouse hepatoma cell invasion and |
| including all | metastasis | metastasis via directly targeting O-GlcNAc |
| different types of | | transferase. Biomed Pharmacother. 91: 731-738. |
| cancers: eg. | | |
| Pancreatic | | |
| cancer | | |
| Anti-tumor | microRNA-24-1 inhibits | Inoguchi et al. (2014). Tumour suppressive |
| treatments | cancer cell proliferation. | microRNA-24-1 inhibits cancer cell |
| including all | | proliferation through targeting FOXM1 in |
| different types of | | bladder cancer. FEBS Lett. 588: 3170-9 |
| cancers: eg. | | |
| Bladder cancer | | |
| Anti-tumor | miR-512-P contributes to | Zhu et al. (2015). Inhibition of RAC1-GEF |
| treatments | suppression of metastasis in | DOCK3 by miR-512-3p contributes to |
| including all | non-small cell lung cancer | suppression of metastasis in non small cell |
| different types of | | lung cancer. Int. J. Biochem Cell Biol. |
| cancers: eg. | | 61: 103-14. |
| Small lung | | |
| cancer | | |
| Anti-tumor | miR-141 inhibits | Kim et al. (2018). Tumor-suppressing miR- |
| treatments | heptacocellular carcinoma | 141-complex loaded tissue-adhesive glue |
| including all | | for the locoregional treatment for |
| different types of | | hepatocellular carcinoma |
| cancers: eg. | | |
| Hepatocellular | | |
| carcinoma | | |
| Anti-tumor | Mir-141-3p suppress tumor | Fang et al (2018). MiR-141-3p suppresses |
| treatments | growth and metastasis | tumor growth and metastasis in Papillary |
| including all | | thyroid cancer via targeting Yin Yang 1. |
| different types of | | Anat Rec (Hoboken). Doi. 10.1002/ar. |
| cancers: eg. | | 23940. |
| Papillary thyroid | | |
| cancer | | |
| Anti-tumor | Mir-141-3p suppress the | Wang et al. (2108). miR-141-3p is a key |
| treatments | growth and migration of | negative regulator of the EGFR pathway in |
| including all | osteosarcoma cells. | osteosarcoma. Onco Targets Ther. 11: 4461-4478. |
| different types of | | |
| cancers: eg. | | |
| Papillary thyroid | | |
| cancer | | |
| Anti-tumor | Mir-148a suppress the | Liu et al. (2018). Long non-coading RNA |
| treatments | growth and migration of | CCAT1/miR-148a/PKCzeta prevents cell |
| including all | prostate cancer | migration of prostate cancer by altering |
| different types of | | macrophage polarization. Prostate. |
| cancers: eg. | | Doi: 10.1002/pro.23716. |
| Papillary thyroid | | |
| cancer | | |
| Other | | |
| Indication | | |
| Targets of pExo | | |
| Wound healing | pExo contains high IL-8. | |
| | IL-8, also known as | |
| | neutrophil chemotactic | |
| | factor, has two primary | |
| | functions. It induces | |
| | chemotaxis in target cells, | |
| | primarily neutrophils but | |
| | also other granulocytes, | |
| | causing them to migrate | |
| | toward the site of infection. | |
| | IL-8 also stimulates | |
| | phagocytosis once they have | |
| | arrived. IL-8 is also known | |
| | to be a potent promoter of | |
| | angiogenesis. In target cells, | |
| | IL-8 induces a series of | |
| | physiological responses | |
| | required for migration and | |
| | phagocytosis, such as | |
| | increases in intracellular | |
| | Ca2+, exocytosis (e.g. | |
| | histamine release), and the | |
| | respiratory burst. | |
| Wound healing | pExo contains PDGF- | |
| | AA/BB: Platelet-derived | |
| | growth factor (PDGF) is | |
| | one of numerous growth | |
| | factors that regulate cell | |
| | growth and division. In | |
| | particular, PDGF plays a | |
| | significant role in blood | |
| | vessel formation, the growth | |
| | of blood vessels from | |
| | already-existing blood | |
| | vessel tissue, mitogenesis, | |
| | i.e. proliferation, of | |
| | mesenchymal cells such as | |
| | fibroblasts, osteoblasts, | |
| | tenocytes, vascular smooth | |
| | muscle cells and | |
| | mesenchymal stem cells as | |
| | well as chemotaxis, the | |
| | directed migration, of | |
| | mesenchymal cells. Platelet- | |
| | derived growth factor is a | |
| | dimeric glycoprotein that | |
| | can be composed of two A | |
| | subunits (PDGF-AA), two | |
| | B subunits (PDGF-BB), or | |
| | one of each (PDGF-AB). | |
| | PDGF is a potent mitogen | |
| | for cells of mesenchymal | |
| | origin, including fibroblasts, | |
| | smooth muscle cells and | |
| | glial cells. In both mouse | |
| | and human, the PDGF | |
| | signalling network consists | |
| | of five ligands, PDGF-AA | |
| | through-DD (including- | |
| | AB), and two receptors, | |
| | PDGFRalpha and | |
| | PDGFRbeta. All PDGFs | |
| | function as secreted, | |
| | disulphide-linked | |
| | homodimers, but only | |
| | PDGFA and B can form | |
| | functional heterodimers | |
| Anti- | pExo contains IL-1RA. IL- | |
| inflamamation | 1RA is a member of the | |
| | interleukin 1 cytokine | |
| | family. IL1Ra is secreted by | |
| | various types of cells | |
| | including immune cells, | |
| | epithelial cells, and | |
| | adipocytes, and is a natural | |
| | inhibitor of the pro- | |
| | inflammatory effect of | |
| | IL1β. This protein inhibits | |
| | the activities of interleukin | |
| | 1, alpha (IL1A) and | |
| | interleukin 1, beta (IL1B), | |
| | and modulates a variety of | |
| | interleukin 1 related | |
| | immune and inflammatory | |
| | responses. | |
| Anti infection, | pExo contains high level of | |
| anti HIV, anti | RANTES (CCL5). CCL5 is | |
| virus infection, | an 8 kDa protein classified | |
| enhance ment of | as a chemotactic cytokine or | |
| NK cell | chemokine. CCL5 is | |
| cytotoxicity | chemotactic for T cells, | |
| | eosinophils, and basophils, | |
| | and plays an active role in | |
| | recruiting leukocytes into | |
| | inflammatory sites. With the | |
| | help of particular cytokines | |
| | (i.e., IL-2 and IFN-γ) that | |
| | are released by T cells, | |
| | CCL5 also induces the | |
| | proliferation and activation | |
| | of certain natural-killer | |
| | (NK) cells to form CHAK | |
| | (CC-Chemokine-activated | |
| | killer) cells. It is also an | |
| | HIV-suppressive factor | |
| | released from CD8+ T cells. | |

### EQUIVALENTS

The present disclosure is not to be limited in scope by the specific embodiments described herein. Indeed, various modifications of the subject matter provided herein, in addition to those described, will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

What is claimed:

1. A method of exosome isolation from a placenta or a portion thereof, the method comprising:
a) contacting a placenta or a portion thereof, preferably cultured placenta or a portion thereof, with a first medium; and
b) obtaining a first fraction comprising a population of exosomes from said placenta or portion thereof;
c) optionally, contacting said placenta or portion thereof with a second medium and obtaining a second fraction comprising a population of exosomes from said placenta or portion thereof;
d) optionally, contacting said placenta or portion thereof with a third medium and obtaining a third fraction comprising a population of exosomes from said placenta or portion thereof; and
e) optionally, isolating the population of exosomes from said first, second, and/or third fractions, preferably by sequential centrifugation and/or affinity chromatography using antibodies or a binding portion thereof specific for a marker or peptide present on a desired population of exosomes, wherein said antibodies or a binding portion thereof are immobilized on a substrate such as a membrane, a resin, a bead, or a vessel.

2. The method of claim 1, wherein the placenta or portion thereof further comprises amniotic membrane.

3. The method of claim 2, wherein the placenta or a portion thereof is a human placenta or a portion thereof.

4.-18. (canceled)

19. The method of claim 1, wherein the third medium comprises a chelator.

20. (canceled)

21. The method of claim 19, wherein the chelator is EDTA or EGTA or a combination thereof.

22.-40. (canceled)

41. The method of claim 1, wherein the exosomes are isolated from said first, second, and/or third fractions or multiple fractions by a method comprising:
(a) passing the first, second and/or third fractions or multiple fractions through a tissue filter;
(b) performing a first centrifugation of the filtrate collected in (a) to generate a cell pellet and a first supernatant;
(c) performing a second centrifugation on the first supernatant to generate a second supernatant; and
(d) performing a third centrifugation on the second supernatant to generate an exosome pellet; and, optionally,
(e) resuspending the exosomes in a solution.

42. The method of claim 1, wherein the exosomes comprise CD63, CD63-A, perforin, Fas, TRAIL or granzyme B or any combination thereof.

43.-47. (canceled)

48. A composition comprising exosomes derived from human placenta, wherein said exosomes are positive for CD1c, CD20, CD24, CD25, CD29, CD2, CD3, CD8, CD9, CD11c, CD14, CD19, CD31, CD40, CD41b, CD42a, CD44, CD45, CD49e, CD4, CD56, CD62P, CD63, CD69, CD81, CD86, CD105, CD133-1, CD142, CD146, CD209, CD326, HLA-ABC, HLA-DRDPDQ, MCSP, ROR1, SSEA-4, or combinations thereof.

49.-50. (canceled)

51. The composition of claim 48, wherein said exosomes are CD3-, CD11b-, CD14-, CD19-, CD33-, CD192-, HLA-A-, HLA-B-, HLA-C-, HLA-DR-, CD11c- or CD34-.

52. (canceled)

53. The composition of claim 48, wherein said exosomes comprise non-coding RNA molecules.

54. The composition of claim 53, wherein said RNA molecules are microRNAs.

55. (canceled)

56. The composition of claim 54, wherein said microRNAs are selected from the group consisting of hsa-mir-26b, hsa-miR-26b-5p, hsa-mir-26a-2, hsa-mir-26a-1, hsa-miR-26a-5p, hsa-mir-30d, hsa-miR-30d-5p, hsa-mir-100, hsa-miR-100-5p, hsa-mir-21, hsa-miR-21-5p, hsa-mir-22, hsa-miR-22-3p, hsa-mir-99b, hsa-miR-99b-5p, hsa-mir-181a-2, hsa-mir-181a-1, hsa-miR-181a-5p, and combinations thereof.

57. The composition of claim 48, wherein said exosomes comprise a cytokine selected from the group consisting of the cytokines in Table 3, and combinations thereof.

58. The composition of claim 48, wherein said exosomes comprise a cytokine receptor selected from the group consisting of the cytokine receptors in Table 4, and combinations thereof.

59. The composition of claim 48, wherein said exosomes comprise a protein selected from the group consisting of the proteins in Table 6, and combinations thereof.

60. The composition of claim 48, wherein said exosomes comprise a protein selected from the group consisting of Cytoplasmic aconitate hydratase, Cell surface glycoprotein MUC18, Protein arginine N-methyltransferase 1, Guanine nucleotide-binding protein G(s) subunit alpha, Cullin-5, Calcium-binding protein 39, Glucosidase 2 subunit beta, Chloride intracellular channel protein 5, Semaphorin-3B, 60S ribosomal protein L22, Spliceosome RNA helicase DDX39B, Transcriptional activator protein Pur-alpha, Programmed cell death protein 10, BRO1 domain-containing protein BROX, Kynurenine—oxoglutarate transaminase 3, Laminin subunit alpha-5, ATP-binding cassette sub-family E member 1, Syntaxin-binding protein 3, Proteasome subunit beta type-7, and combinations thereof.

61. The composition of claim 48, wherein said exosomes comprise at least one marker molecule at a level at least two-fold higher than exosomes derived from mesenchymal stem cells, cord blood, or placental perfusate.

62.-74. (canceled)

75. A method of angiogenesis or vascularization in said subject comprising administering the composition of claim 48 to the subject.

76.-78. (canceled)

79. The method of claim 75, wherein said subject is human.
